SEQUENCE LISTING (2 sequences)

SEQ ID NO: 1 - RNA, 21 nt, Homo sapiens
  source 1..21: mol_type = other RNA
ggttagggcgaaatggtgttt

SEQ ID NO: 2 - RNA, 21 nt, Homo sapiens
  source 1..21: mol_type = other RNA
ggagattaccttacggaaatt

(19) United States
(12) Patent Application Publication
(10) Pub. No.: US 2024/0409944 A1
(43) Pub. Date: Dec. 12, 2024

(54) COMPOSITIONS AND METHODS FOR THE INHIBITION OF TUMOR METASTASIS AND HORIZONTAL GENE TRANSFER

(71) Applicant: The Regents of the University of Colorado, a body corporate, Denver, CO (US)

(72) Inventors: Xuedong Liu, Niwot, CO (US); Quanbin Xu, Boulder, CO (US); Xiaojuan Zhang, Boulder, CO (US); Ashley Jung, Aurora, CO (US); Erica Sung, Buford, GA (US)

(21) Appl. No.: 18/665,838

(22) Filed: May 16, 2024

Related U.S. Application Data
(63) Continuation-in-part of application No. PCT/US2022/079982, filed on Nov. 16, 2022.
(60) Provisional application No. 63/279,780, filed on Nov. 16, 2021.
(60) Provisional application No. 63/283,584, filed on Nov. 29, 2021.

(51) Int. Cl. C12N 15/113

(52) U.S. Cl. CPC C12N 15/1137; C12N 2310/14; C12N 2310/531

(57) ABSTRACT

The invention describes novel systems and methods for cell-to-cell HGT. In one preferred embodiment, a donor cell and a recipient cell are co-cultured, or otherwise brought into contact such that the donor cell and the recipient cell form a cell-to-cell contact to facilitate HGT. In this aspect of the invention, the donor cell is entrapped by the recipient cell forming an intercellular mosaic structure that facilitates the transfer of genetic material from the donor to recipient cell. The invention further described novel systems and methods for blocking cell entrapment and HGT. Such novel systems and methods for blocking cell entrapment and HGT may be used as a treatment for cancer, and in particular may be directed to the prevention of metastasis of cancerous tumors.

### CROSS-REFERENCE TO RELATED APPLICATIONS

This U.S. bypass continuation-in-part claims the benefit of and priority to International PCT application PCT/US2022/079982, filed Nov. 16, 2022, which claims the benefit of and priority to U.S. Provisional Application No. 63/279,780, filed Nov. 16, 2021, and U.S. Provisional Application No. 63/283,584, filed Nov. 29, 2021, and. The entire specification and figures of the above-referenced applications are hereby incorporated, in their entirety, by reference.

### STATEMENT OF FEDERALLY SPONSORED RESEARCH

This invention was made with government support under grant number R01GM113141 awarded by the National Institutes of Health. The government has certain rights in the invention.

### SEQUENCE LISTING

The instant application contains contents of the electronic sequence listing (90245.00693-Sequence-Listing.xml; Size: 2,890 bytes; and Date of Creation: May 16, 2024) is herein incorporated by reference in its entirety.

### TECHNICAL FIELD

The present invention relates to the field of cell regulation and intercellular transfer of genetic material, and specifically novel methods of regulating cell division and horizontal gene transfer via cell entrapment, also referred to herein as tangocytosis. In a preferred embodiment, inhibition of these cellular process may be used to inhibit tumor formation and metastasis in cancer patients.

### BACKGROUND

The transfer of genetic information between different cells and organisms, known as horizontal gene transfer (HGT), is one of the key mechanisms for acquiring new functions during the evolution of prokaryotic and eukaryotic genomes. The mode and consequences of HGT are well established for prokaryotes and fungi. Still, the evidence for cell-contact dependent HGT between different cell types of mammalian cells that result in persistent changes remains scarce due to evolutionary barriers. HGT is also implicated in mechanisms of metastasis in certain cancers. As such, there is a long-felt need for a more complete and comprehensive understanding behind the mechanisms and regulation of cell-to-cell HGT, and in particular cell-to-cell HGT between mammalian cells resulting from cell entrapment.

### SUMMARY OF THE INVENTION

One aspect of the invention includes novel systems and methods for cell-to-cell HGT. In one preferred embodiment, a donor cell and a recipient cell are co-cultured, or otherwise brought into contact such that the donor cell and the recipient cell form a cell-to-cell contact to facilitate HGT. In this aspect of the invention, the donor cell is entrapped by the recipient cell forming an intercellular mosaic structure that facilitates the transfer of genetic material from the donor to recipient cell.

In one aspect the invention includes novel systems, methods and compositions for inhibiting cell entrapment and HGT between donor and recipient cells wherein the donor cell and recipient cells are capable of forming an intercellular mosaic structure resulting in the entrapment of the donor cell by the recipient cell and facilitating cell-to-cell HGT.

In one preferred aspect, cell entrapment and HGT may be decreased by inhibiting the action of ROCK1 or ROCK1/2 or RAP1GDS1 (aka SmgGDS) in the recipient cell. Inhibition of ROCK1 or ROCK1/2 prevents formation of an intercellular mosaic structure mediated by ROCK kinase-dependent actin rearrangement in the recipient cell. In another preferred aspect, cell entrapment and HGT may be decreased by inhibiting actin polymerization in the recipient cell which prevents formation of an intercellular mosaic structure mediated by actin rearrangement in said recipient cell, which further prevents HGT between donor and recipient cells. In another preferred aspect, cell entrapment and HGT may be decreased by inhibiting CDC42 activity in the donor and recipient cells, which prevents formation of an intercellular mosaic structure, which further prevents HGT between donor and recipient cells.

Additional aspects of the invention may include systems, methods, and compositions of treating cancer in a subject in need thereof, and in particular inhibiting metastasis of tumors in a subject. In a preferred aspect, a subject may have a donor tumor cell, in contact with an recipient cell wherein the donor tumor cell and the recipient cell are capable of forming an intercellular mosaic structure resulting in the entrapment of the donor tumor cell by said recipient cell. In this aspect, a subject may be administered a therapeutically effective amount of a target inhibitor that inhibits formation of the intercellular mosaic structure and/or HGT between donor and recipient cells of said subject. Inhibiting of the formation of the intercellular mosaic structure my prevent metastasis of a tumor by preventing escape of the cancer donor cell from the tumor and into surrounding tumor into the surrounding cells/tissue through cell entrapment as described herein.

Another aspect of the invention may include a high-throughput assay for the identification of one or more target inhibitors that prevent the formation of an intercellular mosaic structure resulting in the entrapment of a donor cell by a recipient cell, and the resultant HGT between the cells. In one preferred aspect, donor cells and recipient cells are brought into contact, preferably through co-culturing the cells together. At least one target inhibitor may be introduced to the co-culture of donor and recipient cells, after which the levels and/or rate of entrapment of donor cells by recipient cells forming an intercellular mosaic structure, or the levels and/or rate of transfer of genetic material from donor to recipient cells can be measured and quantified to determine the inhibitors specific activity.

Another aspect of the invention provides a method for treating a disease or disorder, and preferably cancer, comprising administering to a subject in need thereof a therapeutically effective amount of a small molecule compound, or pharmaceutically acceptable salt thereof, that inhibits horizontal gene transfer via cell entrapment, also referred to herein as tangocytosis.

Additional aspects of the invention may be evidenced from the specification, claims and figures provided below.

### BRIEF DESCRIPTION OF DRAWINGS

FIGS. 1A-K: Intercellular gene transfer via cell entrapment. (A) Schematic of the co-culture experiment and the confocal images of RPE1-Venus-Parkin, MDA-MB-231-H2B-mCherry, and a recipient positive for both Venus-Parkin and H2B-mCherry signals. (B) Flow cytometric analysis of RPE1 Venus-Parkin, MDA-MB-231-H2B-mCherry, and recipient cells showing both Venus-Parkin and H2B-mCherry signals. (C) An integration site of MoLV reporter transgene in the genome of recipient cell RPE1mut231. (D) Effect of the donor to recipient cell ratio (Rd/r) on gene transfer frequency. The donor to recipient cell ratio (Rd/r) was calculated as the number of donor cells (Q3+Q4) divided by the number of recipient cells (Q1+Q2). (E) Flow cytometric analysis of gene transfer between RPE1-Venus-Parkin and MDA MB-231-H2B-mCherry cells stably expressing shRNA against mCherry or a luciferase gene. The shRNA mCherry, Rd/r=0.76±0.06; shRNA control, Rd/r=0.53±0.03. (F) Confocal images of the cell-in-cell structure formed by RPE1-Venus-Parkin and MDA-MB-231-H2Bm-Cherry. (G) Confocal live-cell imaging of cell entrapment between RPE1 and MDA-MB-231-H2B-mCherry/CAAX-mCherry. The upper panel shows the engulfment of a donor cell by the recipient cell; the bottom panel shows the exit of a donor cell from the recipient cell. (H) Flow cytometric analysis of gene transfer between MDA-MB-231-H2B-mCherry and RPE1-Venus-Parkin with siRNA against luciferase, ROCK1, ROCK2, or ROCK1 plus ROCK2. (1) Effect of ROCK kinase depletion on cell entrapment between MDA-MB-231-H2B-mCherry and RPE1-Venus-Parkin with siRNA against luciferase, ROCK1, ROCK2, or ROCK1 plus ROCK2. (J-K) Effect of Latrunculin B on intercellular gene transfer and F-actin stability (Alexa Fluor™ 594 Phalloidin). Data are mean±SD; statistical significance for (E and I) was assessed using the student's 1227 test (**** p<0.0001).

FIGS. 2A-H: Characterization of the identity of cells with intercellular gene transfer. (A) Karyotype analysis of RPE1 and RPE1mut231 (n=10). (B) The percentage of transduced cells at different time points of co-culturing. (C-D) Validation of RPE1mut231cells after isolation from co-cultured cells and subsequent passages (P2, P6, P10, and P20). RPE1-Venus-Parkin cells (R1) were co-cultured with MDA-MB-231-H2B-mCherry (M) for 0 and 48 hrs. Cells positive for both Venus-Parkin and H2B-mCherry signals were sorted and subjected to flow cytometry analysis (b) or Western blot analysis (c). (E) PCR amplification of TP53, Venus, and mCherry gene fragments from the genomic DNA of MDA-MB-231-H2B-mCherry (MDA), RPE1VP (RPE1-Venus-Parkin), or RPE1mut231. (F) Scheme of the integration sites of MoLV reporter transgene in RPE1mut231 and MDA-MB-231-H2B-mCherry. (G) The integration site of MoLV reporter transgene in MDA90 MB-231-H2B-mCherry lands in Chromosome 11. One integration site of MoLV reporter transgene in RPE1mut231 was mapped on Chromosome 7. (H) Validation of the integration site in MDA-MB 231-H2B-mCherry (MDA) and RPE1mut231 (Rm). The arrow shows the specific PCR product known as 3C2ndPCR (the integration site junctional fragment) exists only in RPE1mut231 cells; *, non-specific DNA amplification product; RPE, RPE1-Venus-Parkin.

FIGS. 3A-F: Reverse transcription is required for the intercellular transfer of reporter gene. (A) Flow cytometric analysis of the intercellular gene transfer between RPE1-Venus-Parkin (RPE1VP) and MDA-MB-231-H2B-mCherry (MDA231-H2BmCherry) with mCherry mRNA depletion using stably expressed shRNA against luciferase (shLuc) or mCherry (shmCherry). (B) Statistical analysis of mCherry RNA knockdown efficiency in MDA-231-H2B-mCherry cells. (C) qPCR Quantification of mCherry mRNA levels in the MDA-MB-231-H2B mCherry cells stably expressing shRNA against firefly luciferase (shLuc) or mCherry (shmCherry). Untreated RPE1mut231 was used as a control. (D) Statistical analysis of the effect of Stavudine on gene transfer between RPE1-Venus-Parkin and MDA-MB-104 231-H2B-mCherry (one-way ANOVA, p<0.0001). The Rd/r ratios range from 0.64 to 1.11, which have no statistical effect on gene transfer between the recipient and donor cells (Post Hoc analysis, p=0.0618). (E) Effects of Stavudine on the reverse transcriptase activity in cell lysates from the donor and recipient cells. (F) Effect of Stavudine on the cell proliferation of RPE1-Venus-Parkin and MDA-109-MB-231-H2B-mCherry. Data are mean±SD; statistical significance for (B, C and E) was assessed using the student's t-test (*** p<0.001, **** p<0.0001).

FIGS. 4A-F: Direct cell-cell interaction is indispensable for intercellular gene transfer. (A) Semi-coculture assay. This assay allows two types of cells separated physically but still connected by the same medium. Briefly, 1% of percent of agarose in complete DMEM was casted into a 60 mm dishes; holes were made using a 15 ml conical tube after agarose solidified. RPE1-Venus-Parkin and MDA-MB-231-H2B-mCherry were then seeded into left and right holes respectively overnight. Then a bridge between two holes was created using a sterilized blade. (B) Statistical analysis of gene transfer between cells in semi-coculture assay. (C, D) Schematic of experimental design for the effect of vesicles on gene transfer and the corresponding flow cytometric results. The media harvested from MDA-MB-231-H2BmCherry was centrifuged by 2000 rpm to remove cell debris; the vesicles in the supernatant were further collected by 100,000×g centrifugation and then divided into two fractions; one was labeled with Ruby Red dye, and another was left untreated. These two samples were then co-cultured with RPE1-Venus-Parkin cells and followed by flow cytometric analysis. (E) Flow cytometry analysis and quantification of gene transfer between extracellular vesicles labeled or unlabeled and RPE1-Venus-Parkin cells. (F) Flow cytometric results of RPE1-Venus-Parkin or MDA-MB-231-H2B-mCherry culture separated by Transwell insert or co-cultured. Schematic of experimental design (upper panel) and a representative of flow cytometric result (lower panel). Data are mean 129+SD; statistical significance for (Band E) was assessed using a student's t-test (**** p<0.0001).

FIGS. 5A-D: Entrapment of MDA-MB-231 cells by RPE1 cells. (A) Confocal images of coculture of RPE1-Venus-Parkin and MDA-MB-231-H2B-mCherry. These two types of cells were co-cultured for 12 hrs and then subjected to confocal analysis. (B) Confocal images of RPE1-Venus-Parkin and HeLa-H2B-mCherry which were co-cultured for 12 hrs before imaging. (C) Confocal images of coculture of RPE1-Venus-Parkin and MDA-MB-231-H2B-mCherry/CAAX-mCherry. (D) Live cell imaging of RPE1-Venus-Parkin and MDA-MB-231-H2B-mCherry/TagBFP. The nuclei of both cells were labeled with DRAQ5 (pink).

FIGS. 6A-H: ROCK kinases affect cell entrapment and intercellular gene transfer. (A) Flow cytometric analysis of gene transfer between RPE1-Venus-Parkin and MDA-MB-231-H2B141-mCherry with siRNA against luciferase, ROCK1, ROCK2, or ROCK1 plus ROCK2. (B) Western blots show the levels of ROCK1 or 2 in donor and recipient cells with siRNA targeting ROCK1 (K1), ROCK2 (K2), or both (K1&2). (C) Effect of ROCK kinase inhibitor Y27632 on gene transfer. (D) Effect of ROCK kinases inhibition on cell-in-cell structure formation (100 cells/timepoint). (E-H) Effect of the ROCK kinases knockdown or inhibition on the cell proliferation and motility of RPE1-Venus-Parkin (RPE1VP) and MDA-MB-231-H2BmCherry (MDA231-H2BmCh) cells. Data are mean±SD; statistical significance for H was assessed using a one-way ANOVA analysis (**** p<0.0001); ns, not significant.

FIGS. 7A-F: The effect of CDC42 GTPase inhibitor ML141 on gene transfer. (A) RPE1 cells were cocultured with MDA-MB-231-H2B-mCherry (MDA) cells in the presence of ML141. ML141 shows a significant effect on gene transfer (p<0.0001). The inlet shows that the Rd/r ranges from 0.8 to 1.1, which doesn't affect gene transfer significantly based on Post Hoc analysis (p>0.05). (B) The list of genes screened for potential targets affecting gene transfer. The donor or recipient cells expressed validated shRNA or siRNA were co-cultured with the corresponding recipient or donor cells with siRNA/shRNA against luciferase. The gene transfer ratios were analyzed using flow cytometry analysis. The fold change of gene transfer ratio was given by the gene transfer ratio of the donor or recipient cells with specific shRNA/siRNA were divided by the ratio of donor cells and recipient cells with control siRNA. (C) Effect of cell cycle on gene transfer. The donor or recipient cells at the indicated cell cycle stage were co-cultured and the gene transfer ratio was measured using flow cytometry. Asyn, asynchronized cells. (D) Flow cytometry analysis of RPE1-Venus-Parkin co-cultured with MDA-MB-231-H2B-mCherry (MDA231-H2BmCh), MDA-MB-231-αTubulin-mCherry (MDA-TubmCh), or HeLa-H2B-mCherry (HeLa-H2BmCh). (E) Cell type specificity of gene transfer. D. cells, Donor cells; R. cells, Recipient cells; n/a, not available. All donor cell lines stably express H2B-mCherry; SW480 and MCF7 consistently express YFP Mps1. (F) Flow cytometry analysis of coculture of RPE1-LAP-Mps1AS cells (expressing fused GFP-Mps1 protein) and MDA-MB-231-H2B-mCherry (MDA231-H2BmCh) cells. Data are mean±SD; statistical significance for D was assessed using the student's t-test (**** p<0.0001).

FIGS. 8A-C: RPE1 can internalize breast cancer cells which mimics myoepithelial cell and HUVEC cells. (A) Coculture of HUVEC-VenusParkin and MDA-MB-231-H2BmCherry. Inlet shows a HUVEC cells with transferred H2BmCherry; (B) Cell specificity of gene transfer between RPE1, HUVEC and tumor cell lines; (C) 3D culture of RPE1-VenusParkin with MDA-MB-231-H2BmCherry

FIGS. 9A-D: Tangocytosis inhibition impairs TNBC cells migration and transmigration in vitro. (A) Scratch assay for MDA231-h2bmcherry/BFP with drugs as indicated; (B) Scheme of cell transmigration assay; (C) presentative image of transmigration assay; * shows a MDA cells move to the bottom through transcellular pathway; (D) statistical result for flow cytometry.

FIGS. 10A-C: High content screening for inhibitors for tangocytosis.

FIGS. 11A-B: HCT identified potential tangocytosis inhibitors and targets by grouping.

FIG. 12. Representative results of exemplary CICs inhibitors.

FIGS. 13A-C: DNA damaging drugs are potential tangocytosis inhibitors. (A-C) Growth inhibition of DNA damaging drugs (Clofarabine, Teniposide, and Pemetrexed) on RPE1 and MDA231 cells.

FIGS. 14A-C: DNA damages drugs block gene transfer. (A-C) Gene transfer inhibition by adding DNA damaging hits simultaneously.

FIGS. 15A-F: Pretreatment of RPE1 or MDA cell's DNA damages drugs have opposite effects on gene transfer.

FIGS. 16A-C: Cell-in-Cell structure can be frozen by Tangocytosis inhibitors Doxorubicin and Actinomycin.

FIGS. 17A-B: RhoA/ROCKs pathway negatively regulates Tangocytosis.

FIG. 18A-B: Co-cultured RPE1-VEP and MDA-MB-231-H2B-mCherry were treated with 10 μM, 20 μM, and 40 μM concentrations of Actinomycin D and Doxorubicin and the frequency of CIC structures were quantified every 6 hours during 48 hour live cell imaging.

FIG. 19: The cell entry and exit were visualized with 48-hour live cell imaging. The images show a cell-in-cell structure formation with 20 μM Actinomycin D treatment. MDA-MB-231 cell dived into RPE1 cell at 8 hours and 20 minutes and exited at 13 hours and 20 minutes.

FIG. 20A-E. A Labeled flow cytometry analysis showing RPE1-VenusParkin, MDA-MB-231-H2BmCherry, and a double positive cell expressing both VenusParkin and H2b-mCherry B Flow cytometry analysis showing a comparison between DMSO vs. 100 μM Hygromycin B; Hygromycin B enhances gene transfer C 72 hour Co-cultured RPE1-VEP and MDA-MB-231-H2B-mCherry were treated with 50 μM, 100 μM, and 200 μM concentrations of Hygromycin B and the gene transfer % was quantified using flow cytometry D Co-culturedRPE1-VEP and MDA-MB-231-H2B-mCherry were treated with DMSO and 100 μM and Hygromycin B and flow cytometry was used to quantify H2B-mCherry gene expression in each treatment E Flow Cytometry Analysis representing of Hygromycin B's effect on H2B-mCherry gene expression.

FIG. 21: Entry and Exit Time recorded for CIC structures in each antibiotic treatment.

FIG. 22: Gene transfer percentage obtained from flow cytometry results of DMSO, 20 μM Actinomycin D, 100 μM Hygromycin B, and both treatments together.

FIG. 23A-B: A Duration of CIC structures in each drug treatment was averaged and compared with each other B Frequency of CIC structures was recorded for each drug treatment.

FIG. 24A-B: A RPE1-VEP and MDASC43 co-cultured cells were treated with DMSO, 10 μM Doxorubicin, and 20 μM Actinomycin D, 100 μM Hygromycin B, 10 μM Doxorubicin and 100 μM Hygromycin B, and 20 μM Actinomycin D and Hygromycin B. The entry and exit times of each CIC structure were recorded from 48-hour live cell imaging B The number of CIC structures was quantified for each antibiotic treatment.

FIG. 25: Average duration of CIC structures in each drug treatment.

FIG. 26A-B: (A,B) Schematic representation after cell sorting, showing VenusParkin, H2BmCherry, and double positive gene expression “RPE1mut231.”

FIG. 27A-B: A Area Pixels\hat{ } 2/6 hours determined from “Wound Healing Size Tool” ImageJ plugin of each cell line of the parental, CIC, and RPE1mut231. B Crystal Violet staining was performed over 5 days and absorbance at 570 nm was measured with Spectramax.

FIG. 28A-C: Wound healing comparisons between A Parental vs. CIC MDA-MB-231-H2B-mCherry B Parental vs. CIC RPE1 C RPE1mut231.

FIG. 29: Co-cultured Parental MDA-MB-231-H2B-mCherry & Parental RPE1-VEP, CIC RPE & CIC MDA, and MDASC43 & RPE1-VEP were each treated with 100 μM Hygromycin B for 48 hours. Gene transfer percentage was calculated using flow cytometry and compared with DMSO control.

FIG. 30: Average entry and exit time of MDA-MB-231-H2B-mCherry in HUVEC cells for each antibiotic treatment.

FIG. 31: Depiction of Hygromycin B and Doxorubicin's role in cell entry and exit during cell-in-cell structure formation.

FIG. 32: Schematic of the potential role of Actinomycin D, Doxorubicin, Hygromycin B, and Stavudine on the gene transfer mechanism from tumor cell to recipient cell.

FIG. 33: Chemical structure of Hygromycin B.

### DETAILED DESCRIPTION OF THE INVENTION

The inventive technology includes novel systems, methods, and compositions or for regulating cell-to-cell entrapment and HGT. In one preferred embodiment, a donor cell and a recipient cell are co-cultured, or otherwise brought into contact such that the donor cell and recipient cell are capable of forming an intercellular mosaic structure resulting in the entrapment of the donor cell by the recipient cell. In this embodiment of the invention, the donor cell is entrapped by the recipient cell forming an intercellular mosaic structure that facilitates the transfer of genetic material from the donor to recipient cell. In this embodiment, the transfer of genetic material may include the transfer of one or more nucleic acids from the entrapped donor cell to the recipient cell. Examples of genetic material to be transferred by this process may include RNA, such as an intermediate mRNA that may be translated, and/or reverse transcribed into a DNA molecule and integrated into the genome of the recipient cell or otherwise expressed.

The invention further includes novel systems, methods, and compositions for regulating cell-to-cell entrapment and HGT between two different cell types, and preferably different mammalian cell types. In one preferred embodiment, a system of cell-to-cell HGT and entrapment may include a donor cell and a recipient cell that may preferably be different cell types that are capable of forming an intercellular mosaic structure resulting in the entrapment of the donor cell by the recipient cell when brought into contact. These cells may be contacted through a process of co-culturing the donor and recipient cells such that all or a portion of the donor cells may be entrapped by corresponding recipient cells forming an intercellular mosaic structure which facilitates the cell-to-cell transfer of genetic material from the donor cell to the recipient cell prior to disengagement of the donor cell by the corresponding recipient cell. As noted above, in some embodiments the genetic material transferred by the donor cell to the recipient cell may include a nucleic acid, such as an intermediate mRNA that may be translated, and/or reverse transcribed into a DNA molecules and further integrated into the genome of the recipient cell for stable heterologous expression.

The invention further includes novel systems, methods, and compositions for regulating cell-to-cell HGT and entrapment between two different cell types, and preferably different mammalian cell types wherein one cell is a tumor or cancer cell, and the recipient is an epithelial cell, or a cell in contact with a cancer or tumor cell. In one preferred embodiment, a system of cell-to-cell HGT and entrapment may include a cancer donor cell, such as a breast cancer cell or similar cancer cell that may be part of a tumor, and an epithelial recipient cell that may be in contact with the donor cancer cell. In this embodiment, the cancer donor cell and the epithelial recipient cell are capable of forming an intercellular mosaic structure resulting in the entrapment of the cancer donor cell by the epithelial recipient cell when brought into contact. A cancer donor cell and epithelial recipient cell may be contacted, in vitro for example through co-culturing the cells together, such that all or a portion of cancer donor cell may be entrapped by corresponding epithelial recipient cell forming an intercellular mosaic structure which facilitates the cell-to-cell transfer of genetic material from the cancer donor cell to said epithelial recipient cell prior to disengagement of the cancer donor cell by the corresponding epithelial recipient cell. I

In this aspect, the intercellular mosaic structure and HGT may be facilitated by actin rearrangement in the recipient cell, and in particular ROCK kinase-dependent actin rearrangement in the recipient cell. As noted above, in some embodiments the genetic material transferred by the cancer donor cell to a recipient cell may include a nucleic acid, such as an intermediate mRNA that may be translated, and/or reverse transcribed into a DNA molecules and further integrated into the genome of the recipient cell for stable heterologous expression or extrachromosomal circular DNA elements (eccDNAs). As also noted above, in certain embodiments, the cancer donor cell may be entrapped by an epithelial cell that may be contacting a tumor. In this configuration, the entrapped cancer cell may be disengaged by the epithelial cell and dissociated from the tumor so as to promote metastasis of the cancer/tumor in a subject.

The invention further includes novel systems, methods, and compositions for regulating cell-to-cell HGT and entrapment between two different cell types, and preferably different mammalian cell types wherein one cell is a tumor or cancer cell, and the recipient cell is an epithelial cell, or a cell in contact with a cancer or tumor cell. In one preferred embodiment, a system of cell-to-cell HGT and entrapment may include a cancer donor cell, such as MDA-MB-231 or similar cancer cell, and a recipient cell, such as a RPE1 cell or a similar epithelial cell that that is capable of forming an intercellular mosaic structure resulting in the entrapment of the MDA-MB-231 donor cell by the RPE1 recipient cell when brought into contact. A MDA-MB-231 donor cell and RPE1 recipient cell may be contacted in vitro, for example through co-culturing the cells together, preferably at a ratio or 1:1, such that all or a portion of MDA-MB-231 donor cell may be entrapped by corresponding RPE1 recipient cell forming an intercellular mosaic structure that facilitates the cell-to-cell transfer of genetic material from the MDA-MB-231 donor cell to the RPE1 recipient cell prior to disengagement of the MDA-MB-231 donor cell by the corresponding RPE1 recipient cell. In this aspect, the intercellular mosaic structure and HGT may be facilitated by actin rearrangement in the RPE1 recipient cell, and in particular ROCK kinase-dependent actin rearrangement in the RPE1 recipient cell. As noted above, in some embodiments the genetic material transferred by the MDA-MB-231 donor cell to the RPE1 recipient cell may include a nucleic acid, such as an intermediate mRNA that may be translated, and/or reverse transcribed into a DNA molecules and further integrated into the genome of the recipient cell for stable heterologous expression.

In one aspect the invention includes novel systems, methods, and compositions for inhibiting cell entrapment and HGT between donor and recipient cells wherein the donor and recipient cells are capable of forming an intercellular mosaic structure resulting in the entrapment of the donor cell by the recipient cell and facilitating cell-to-cell HGT. In one preferred aspect, the cell entrapment and HGT may be decreased by inhibiting the action of ROCK1 or ROCK1/2 or RAP1GDS1/SmgGDS in the recipient cell, which prevents formation of an intercellular mosaic structure mediated by ROCK kinase-dependent actin rearrangement in the recipient cell.

The invention includes systems, methods and compositions for regulating cell entrapment and horizontal gene transfer between cells. As noted above, a donor cell and a recipient cell may establish cell-to-cell contact in vitro or in vivo, thereby initiating ROCK kinase-dependent actin rearrangement in the recipient cell forming an intercellular mosaic structure resulting in the entrapment of the donor cell by the recipient cell. As noted, this cell-to-cell contact may occur in an in vitro, or in vivo environment. For example, cell-to-cell contact of a donor and recipient cell may occur as a result of the co-culturing of the cells together in vitro. Cell-to-cell contact of a donor and recipient cell may also occur in vivo, for example in a subject, wherein a donor cell is positioned in contact with a recipient cell. In a preferred embodiment, the donor cell of the subject is a cancer cell, and preferably part of a tumor, while the recipient cell may include an epithelial cell, or other cell in contact with said cancer cell or surrounding the tumor. In another embodiment, cell-to-cell HGT and entrapment may include a cancer donor cell, such as MDA-MB-231 or similar cancer cell, and a recipient cell, such as RPE1 or a similar epithelial cell that that are capable of forming an intercellular mosaic structure resulting in the entrapment of the MDA-MB-231 donor cell by the RPE1 recipient cell when brought into contact, whether in vitro in a co-culture, or in vivo in a subject, and preferably a subject having cancer.

As noted above, inhibition of ROCK kinase-dependent actin rearrangement in the recipient cell may prevent formation of an intercellular mosaic and HGT between donor and recipient cells. As such, in certain embodiments a ROCK1 or ROCK1/2 inhibitor, RAP1GDS1/SmgGDS or a combination of the same may be used to block or reduce ROCK kinase-dependent actin rearrangement in the recipient cell thereby preventing the formation of an intercellular mosaic structure resulting in the entrapment of the donor cell by the recipient cell. In this embodiment, a ROCK1 or ROCK 1/2 or RAP1GDS1/SmgGDS inhibitor of the invention may include, but not be limited to: a small-molecule, a small-inhibitory RNA (siRNA), a short hairpin RNA (shRNA), a bifunctional RNA, an antisense oligonucleotide, an anti-ROCK1 or ROCK1/2 antibody or functional fragment thereof, a ribozyme, a deoxyribozyme, an aptamer, a small molecule or gene therapy that knocks out ROCK1 or ROCK1/2 or RAP1GDS1/SmgGDS

An inhibitor of ROCK kinase-dependent actin rearrangement may include ROCK kinase inhibitor Y27632 (Y-27632 dihydrochloride), or a pharmaceutical compositions thereof containing a compound according to according to Formula I:

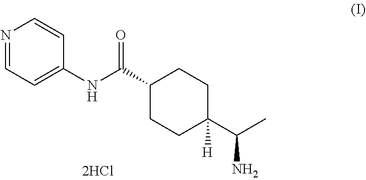 [SMILES: C[C@@H](N)[C@H]1CC[C@H](C(=O)Nc2ccncc2)CC1]

In another embodiment, ROCK kinase-dependent actin rearrangement may be inhibited by RNA interference. For example, an siRNA may be generated to target ROCK1/2, or preferably ROCK1 in the recipient cell and inhibit ROCK kinase-dependent actin rearrangement. In a preferred embodiment, an siRNA of the invention may be configured to inhibit expression of ROCK1 or ROCK2 and comprises as siRNA sequence according to SEQ ID NO. 1, and SEQ ID NO. 2, respectively. As noted above, in a preferred embodiment, an siRNA configured to inhibit expression of ROCK1 according to SEQ ID NO. 1 may be contacted with the recipient cell to inhibit ROCK kinase-dependent actin rearrangement preventing donor cell entrapment and associated HGT as described herein.

As noted above, inhibition of actin polymerization in the recipient cell may prevent formation of an intercellular mosaic structure and HGT between donor and recipient cells. As such, in certain embodiments an actin polymerization inhibitor may be used to block or reduce actin polymerization in the recipient and/or donor cells thereby preventing the formation of an intercellular mosaic structure resulting in the entrapment of the donor cell by the recipient cell. In this embodiment, an actin polymerization inhibitor of the invention may include, but not be limited to: a small-molecule, a small-inhibitory RNA (siRNA), a short hairpin RNA (shRNA), a bifunctional RNA, an antisense oligonucleotide, antibody or functional fragment thereof, a ribozyme, a deoxyribozyme, an aptamer, a small molecule or gene therapy that knocks out one or more gene related to actin polymerization. An actin polymerization inhibitor of the invention may include Latrunculin B, or a pharmaceutical compositions thereof containing a compound according to according to Formula II:

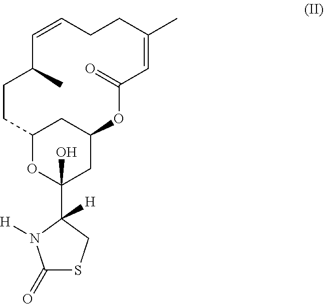

In another preferred aspect, the cell entrapment and HGT may be decreased by inhibiting CDC42 activity or expression in the donor and recipient cells, which prevents formation of an intercellular mosaic structure, which further prevents HGT between donor and recipient cells. As such, in certain embodiments a CDC42 inhibitor may be used to block or reduce a CDC42 in the recipient cell thereby preventing the formation of an intercellular mosaic structure resulting in the entrapment of said donor cell by said recipient cell. In this embodiment, a CDC42 inhibitor of the invention may include, but not be limited to: a small-molecule, a small-inhibitory RNA (siRNA), a short hairpin RNA (shRNA), a bifunctional RNA, an antisense oligonucleotide, anti-CDC42 antibody or functional fragment thereof, a ribozyme, a deoxyribozyme, an aptamer, a small molecule or gene therapy that knocks out one or more gene related to actin polymerization. A CDC42 inhibitor may include ML141, or a pharmaceutical compositions thereof containing a compound according to according to Formula III:

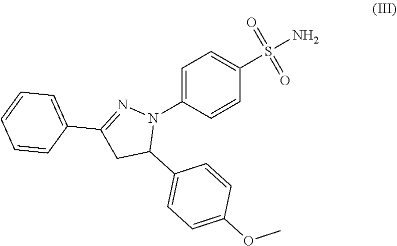 [SMILES: COc1ccc(C2CC(c3ccccc3)=NN2c2ccc(S(N)(=O)=O)cc2)cc1]

The invention may further include systems, methods and compositions of treating cancer in a subject in need thereof, and in particular inhibiting metastasis of cancer cell/tumors in a subject. In a preferred embodiment, a subject may have a donor cancer cell, in contact with an recipient cell, and preferably an epithelial cell in contact with the cancer donor cell or surrounding a tumor, wherein the donor cancer cell and the recipient cell are capable of forming an intercellular mosaic structure resulting in the entrapment of the donor cancer cell by said recipient cell and facilitating HGT from donor to recipient cells. In this aspect, a subject may be administered a therapeutically effective amount of a target inhibitor that inhibits formation of an intercellular mosaic structure and/or HGT between donor and recipient cells of said subject.

In another preferred aspect, the cell entrapment and HGT may be decreased by administering a therapeutically effective amount of a pharmaceutical compositions containing one or more compounds selected from: Clofarabine, Clorprenaline HCL, Nedaplatin, Nitroxoline, Chloroxine, Actinomycin D, Mitomycin C, Ciclopirox ethanolamine, Ciclopirox, VX-680 (MK-0457, Tozasertib), Ponatinib (AP24534), Nisoldipine, Atracurium Besylate, Roscovitine (Seliciclib,CYC202), LEE011, Mycophenolate Mofetil, Mycophenolic acid, Vidofludimus, Methotrexate, Pralatrexate, Pyrimethamine, Pemetrexed, Teriflunomide, Thio-TEPA, Cytarabine hydrochloride, Raltitrexed, VRT752271, Amoxapine, Raltegravir (MK-0518), S/GSK1349572, GSK1349572 sodiuM salt, Amprenavir (agenerase), Atazanavir, Darunavir, Darunavir Ethanolate, Zidovudine, Stavudine (d4T), GS-7340 (Tenofovir), Tenofovir Disoproxil Fumarate, Zalcitabine, Ganetespib (STA-9090), Diacerein, Trametinib (GSK1120212), Trametinib DMSO solvate, Pimasertib (AS-703026), AZD6244 (Selumetinib), Ridaforolimus (Deforolimus, MK-8669), Zotarolimus (ABT-578), Domiphen Bromide, BMN 673, Anagrelide HCl, BI6727 (Volasertib), Hexachlorophene, Pemetrexed disodium hemipenta hydrate, Irinotecan hydrochloride, Etoposide, Irinotecan, Irinotecan HCl Trihydrate, Epirubicin HCl, Doxorubicin, Doxorubicin (Adriamycin) HCl, Sunitinib, Sunitinib malate, Dovitinib Dilactic acid, and Teniposide, or a combination of the same.

A target inhibitor of the invention may include, but not be limited to a ROCK1/2 inhibitor, a ROCK1 inhibitor, an actin polymerization inhibitor, a CDC42 inhibitor, or a combination of the same as generally described herein. In alternative embodiments, a target inhibitor of the invention may be selected from the group consisting of: a small-molecule, a small-inhibitory RNA (siRNA), a short hairpin RNA (shRNA), a bifunctional RNA, an antisense oligonucleotide, an antibody or functional fragment thereof, a ribozyme, a deoxyribozyme, an aptamer, a small molecule, a peptide or gene therapy that knocks out a target gene.

In one embodiment, a target inhibitor of the invention may include, but not be limited to: Clofarabine, Clorprenaline HCL, Nedaplatin, Nitroxoline, Chloroxine, Actinomycin D, Mitomycin C, Ciclopirox ethanolamine, Ciclopirox, VX-680 (MK-0457,Tozasertib), Ponatinib (AP24534), Nisoldipine, Atracurium Besylate, Roscovitine (Seliciclib, CYC202), LEE011, Mycophenolate Mofetil, Mycophenolic acid, Vidofludimus, Methotrexate, Pralatrexate, Pyrimethamine, Pemetrexed, Teriflunomide, Thio-TEPA, Cytarabine hydrochloride, Raltitrexed, VRT752271, Amoxapine, Raltegravir (MK-0518), S/GSK1349572, GSK1349572 sodiuM salt, Amprenavir (agenerase), Atazanavir, Darunavir, Darunavir Ethanolate, Zidovudine, Stavudine (d4T), GS-7340 (Tenofovir), Tenofovir Disoproxil Fumarate, Zalcitabine, Ganetespib (STA-9090), Diacerein, Trametinib (GSK1120212), Trametinib DMSO solvate, Pimasertib (AS-703026), AZD6244 (Selumetinib), Ridaforolimus (Deforolimus, MK-8669), Zotarolimus (ABT-578), Domiphen Bromide, BMN 673, Anagrelide HCl, BI6727 (Volasertib), Hexachlorophene, Pemetrexed disodium hemipenta hydrate, Irinotecan hydrochloride, Etoposide, Irinotecan, Irinotecan HCl Trihydrate, Epirubicin HCl, Doxorubicin, Doxorubicin (Adriamycin) HCl, Sunitinib, Sunitinib malate, Dovitinib Dilactic acid, Teniposide, Y27632, Latrunculin B., and ML141, or a combination of the same.

Another aspect of the invention may include a high-throughput assay for the identification of one or more target inhibitors that prevent the formation of an intercellular mosaic structure resulting in the entrapment of a donor cell by a recipient cell, and the resultant HGT. In one preferred aspect, donor and recipient cells are brought into contact, preferably through co-culturing the cells in vitro. In this example, the donor cell is a MDA-MB-231 donor cell, and said recipient cell is an RPE1 recipient cell that are co-cultured in vitro, and preferably at a ratio of approximately 1:1. At least one target inhibitor may be introduced to the co-culture of donor and recipient cells, after which the levels and/or rate of entrapment of donor cells by recipient cells forming an intercellular mosaic structure, or the levels and/or rate of transfer of genetic material from donor to recipient cells can be measured and quantified to determine the inhibitors specific activity and/or target. A target inhibitor of the invention for use in the described high-throughput assay may include, but not be limited to a ROCK1/2 inhibitor, a ROCK1 inhibitor, an actin polymerization inhibitor, a CDC42 inhibitor, or a combination of the same as generally described herein. Further examples of target inhibitors for use in the described high-throughput assay include one or more target inhibitors selected from the group consisting of: a small-molecule, a small-inhibitory RNA (siRNA), a short hairpin RNA (shRNA), a bifunctional RNA, an antisense oligonucleotide, an antibody or functional fragment thereof, a ribozyme, a deoxyribozyme, an aptamer, a small molecule or gene therapy that knocks out a target gene.

Notably, the transfer of genetic material from a donor to recipient cell may include a nucleic acid encoding a biomarker and/or reporter gene, such as a fluorescent protein or other marker known by those of ordinary skill in the art. Such marker or reporter proteins may be expressed in the recipient cell and measured using flow cytometric analysis to quantify the intercellular transfer, and or through live cell imaging to detect the entrapment of the donor cell by the recipient cell.

As used herein the singular forms “a”, “an”, and “the” include plural referents unless the context clearly dictates otherwise. Thus, for example, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or +a range of up to 20%, up to 15%, up to 10%, up to 5%, or up to 4%, 3%, 2%, or 1% compared to the specifically recited value.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

The term “cancer” means a disease characterized by the pathological proliferation of a cell or tissue and its subsequent migration to or invasion of other tissues or organs. Cancer growth is typically uncontrolled and progressive, and occurs under conditions that would not elicit, or would cause cessation of, multiplication of normal cells. Cancers can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Non-limiting examples of cancers include: acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pincaloma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

The term “metastasis” means the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential.

The term “donor cell” means a cell that transmits genetic material to a recipient cell through cell-to-cell mediated HGT. The term “recipient cell” means a cell that receives genetic material to a donor cell through cell-to-cell mediated HGT.

The term “genetic material” means a nucleic acid as defined herein, and preferably includes a gene or fragment thereof. The term “gene” is meant to refer to a segment of nucleic acid that contains the information necessary to produce a functional RNA product. A gene usually contains regulatory regions dictating under what conditions the RNA product is made, transcribed regions dictating the sequence of the RNA product, and/or other functional sequence regions. A gene may be transcribed to produce an mRNA molecule, which contains the information necessary for translation into the amino acid sequence of the resulting protein.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

The term “reverse transcription” means the generation of a complementary DNA (cDNA) from an RNA template by the enzyme reverse transcriptase.

A “reporter protein” refers to an amino acid sequence that, when present in a cell or tissue, is detectable and distinguishable from other genetic sequences or encoded polypeptides present in cells. A reporter protein may be a naturally occurring protein or a protein that is not naturally occurring. If naturally occurring, it may be detectable as a result of the amount of expression following gene transfer, or it may be a protein to which a detectable tag can be attached. Examples of such reporter proteins include fluorescent proteins such as green fluorescent protein (gfp), cyan fluorescent protein (cfp), red fluorescent protein (rfp), or blue fluorescent protein (bfp), or derivatives of these proteins, or enzymatic proteins such as β-galactosidase, chemilluminesent proteins such as luciferase, somatostatin receptor amino acid sequence, a sodium iodide symporter amino acid sequence, a luciferase amino acid sequence, and a thymidine kinase amino acid sequence.

As used herein, a “biomarker” or “marker” is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process. In a preferred embodiment a biomarker is a peptide, and preferably an amino acid sequence that, when present in a cell or tissue, is detectable and distinguishable from other genetic sequences or encoded polypeptides present in cells.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, “inhibits,” “inhibition” refers to the decrease relative to the normal wild-type level, or control level. Inhibition may result in a decrease, for example of cell-to-cell HGT or tangocytosis, and/or cell entrapment, in response an inhibitor, such as a ROCK1/2 inhibitor, a ROCK1 inhibitor, an actin polymerization inhibitor, a CDC42 inhibitor, or a combination of the same, by less than 10%, 10%, 15%, 20%, 25%, 30%, 35%, 40%, 45%, 50%, 55%, 60%, 65%, 70%, 75%, 80%, 85%, 90%, 95% or 100%, and all ranges described herein.

In one embodiment, a specific gene may be inhibited, such that its level of expression is inhibited. Th terms “levels” or “expression” means the amount of a protein or RNA present in a cell (e.g., a cancer cell or a control cell). In one embodiment, a specific peptide may be inhibited, such that its activity is inhibited. The term “activity” means the level of functionality and/or interaction of a peptide with other molecules within a cell.

As used herein, “a target inhibitor” refers to a compositions that inhibits or prevents cell-to-cell HGT, and/or cell entrapment between a donor and a recipient cell. In a preferred embodiment, a “target inhibitor” may be administered as part of a pharmaceutical composition. “Pharmaceutical compositions” are compositions that include an amount (for example, a unit dosage) of the disclosed compound(s) together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition). In another embodiment, a compound of the invention, and preferably a ROCK1/2 inhibitor, a ROCK1 inhibitor, an actin polymerization inhibitor, a CDC42 inhibitor, or a combination of the same, may be in the form of a pharmaceutically acceptable salt or ester. The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid, and the like.

Such pharmaceutical compositions/formulations are useful for administration to a subject, in vivo or ex vivo. Pharmaceutical compositions and formulations include carriers or excipients for administration to a subject. As used herein the terms “pharmaceutically acceptable” and “physiologically acceptable” mean a biologically compatible formulation, gaseous, liquid, or solid, or mixture thereof, which is suitable for one or more routes of administration, in vivo delivery, or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules, and crystals. Supplementary active compounds (e.g., preservatives, antibacterial, antiviral, and antifungal agents) can also be incorporated into the compositions. The formulations may, for convenience, be prepared or provided as a unit dosage form. In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

Pharmaceutical formulations and delivery systems appropriate for the compositions and methods of the invention are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N. Y., pp. 253-315). For example, pharmaceutical compositions can optionally be formulated to be compatible with a particular route of administration. Exemplary routes of administration include administration to a biological fluid, an immune cell (e.g., T or B cell) or tissue, mucosal cell or tissue (e.g., mouth, buccal cavity, labia, nasopharynx, esophagus, trachea, lung, stomach, small intestine, vagina, rectum, or colon), neural cell or tissue (e.g., ganglia, motor or sensory neurons) or epithelial cell or tissue (e.g., nose, fingers, cars, cornea, conjunctiva, skin or dermis). Thus, pharmaceutical compositions include carriers (excipients, diluents, vehicles, or filling agents) suitable for administration to any cell, tissue, or organ, in vivo, ex vivo (e.g., tissue or organ transplant) or in vitro, by various routes and delivery, locally, regionally, or systemically.

Exemplary routes of administration for contact or in vivo delivery of a target inhibitor, is a dosage of the compound that is sufficient to achieve a desired therapeutic effect, such as can optionally be formulated include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, and intralymphatic.

The term “effective” or “therapeutically effective” is to be understood broadly to include reducing or alleviating the signs or symptoms of cancer, improving the clinical course of cancer, or reducing any other objective or subjective indicia of the cancer, for example through inhibiting metastasdis or preventing tangocytosis and/or HGT. It also includes reducing or alleviating the signs or symptoms of cancer, improving the clinical course of the disease, or reducing any other objective or subjective indicia of the disease. Different drugs, doses and delivery routes can be evaluated by performing the method using different drug administration conditions. The molecular targets may also be used as pharmaceutical compositions or in kits. The targets may also be used to screen candidate compounds that modulate their expression.

In one embodiment, a target inhibitor may inhibit one or more genes through RNA interference. “RNA interference” (RNAi) is meant a phenomenon where double-stranded RNA homologous to a target mRNA leads to degradation of the targeted mRNA (e.g., a ROCK1 mRNA). RNAi is more broadly defined as degradation of target mRNAs by homologous siRNAs. By “siNA” is meant small interfering nucleic acids. One exemplary siNA is composed of ribonucleic acid (siRNA). siRNAs can be 21-25nt RNAs derived from processing of linear double-stranded RNA. siRNAs assemble in complexes termed RISC (RNA-induced silencing complex) and target homologous RNA sequences for endonucleolytic cleavage. Synthetic siRNAs also recruit RISCs and are capable of cleaving homologous RNA sequences.

As used herein, the term “antisense” means a polynucleotide or analog whose sequence of bases is complementary to messenger RNA. As used herein, the term “sense” means a polynucleotide or analog whose sequence of bases is complementary to a messenger RNA.

As used herein, the term “mammal” includes living mammals including living humans and living non-human animals such as murine, porcine, canine, rodentia and feline. The terms “individual,” “subject,” and “patient,” used interchangeably herein, refer to a mammal, including, but not limited to, murines, simians, humans, mammalian farm animals, mammalian sport animals, and mammalian pets. Preferably, the subject herein is human. As used herein, the phrase “in need thereof” means that the animal or mammal has been identified as having a need for the particular method or treatment. In some embodiments, the identification can be by any means of diagnosis.

In any of the methods and treatments described herein, the animal or mammal can be in need thereof. In some embodiments, the animal or mammal is in an environment or will be traveling to an environment in which a particular disease, disorder, or condition is prevalent.

By “treating” a disease, disorder, or condition is meant delaying an initial or subsequent occurrence of a disease, disorder, or condition; increasing the disease-free survival time between the disappearance of a condition and its reoccurrence; stabilizing or reducing one or more (e.g., two, three, four, or five) adverse symptom(s) associated with a condition; or inhibiting, slowing, or stabilizing the progression of a condition. The term “treating” also includes reducing (e.g., by at least 5%, 10%, 15%, 20%, 25%, 30%, 35%, 40%, 45%, 50%, 55%, 60%, 65%, 70%, 75%, 80%, 85%, 90%, or 95% the severity or duration of one or more (e.g., one, two, three, four, or five) symptoms of a disease (e.g., cancer) in a patient. Desirably, at least 20%, 40%, 60%, 80%, 90%, or 95% of the treated subjects have a complete remission in which all evidence of the disease disappears. In another desirable embodiment, the length of time a patient survives after being diagnosed with a condition and treated using the methods of the invention is at least 20%, 40%, 60%, 80%, 100%, 200%, or even 500% greater than (i) the average amount of time an untreated patient survives or (ii) the average amount of time a patient treated with another therapy survives. In the methods of the invention, a target inhibitor, may be administered in accordance with the methods at any frequency as a single bolus or multiple dose e.g., one, two, three, four, five, or more times hourly, daily, weekly, monthly or annually or between about 1 to 10 days, weeks, months, or for as long as appropriate. Exemplary frequencies are typically from 1-7 times, 1-5 times, 1-3 times, 2-times or once, daily, weekly, or monthly. Timing of contact, administration ex vivo or in vivo delivery can be dictated by the pathogenesis, symptom, pathology, or adverse side effect to be treated. For example, an amount can be administered to the subject substantially contemporaneously with, or within about 1-60 minutes or hours of the onset of a symptom or adverse side effect of treatment.

Doses may vary depending upon whether the treatment is therapeutic or prophylactic, the onset, progression, severity, frequency, duration, probability of or susceptibility of the symptom, the type of pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the pathology or symptom, or any adverse side effects of the treatment or therapy. The skilled artisan will appreciate the factors that may influence the dosage, frequency and timing required to provide an amount sufficient or effective for providing a prophylactic or therapeutic effect or benefit.

Doses can be based upon current existing treatment protocols, empirically determined, determined using animal disease models or optionally in human clinical studies. A subject may be administered in single bolus or in divided/metered doses, which can be adjusted to be more or less according to the various consideration set forth herein and known in the art. Dose amount, frequency or duration may be increased or reduced, as indicated by the status of pathogenesis, associated symptom or pathology, or any adverse side effect(s). For example, once control or a particular endpoint is achieved, for example, reducing, decreasing, inhibiting, ameliorating, or preventing onset, severity, duration, progression, frequency, or probability of one or more symptoms associated with a telomere-associated disease or disorder. Another embodiment of this disclosure provides pharmaceutical kits containing a pharmaceutical composition of this disclosure containing a target inhibitor, prescribing information for the composition, and a container.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

Each publication or patent cited herein is incorporated herein by reference in its entirety. The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1: Demonstration of Novel Mode of Intercellular Gene Transfer in Mammalian Cells

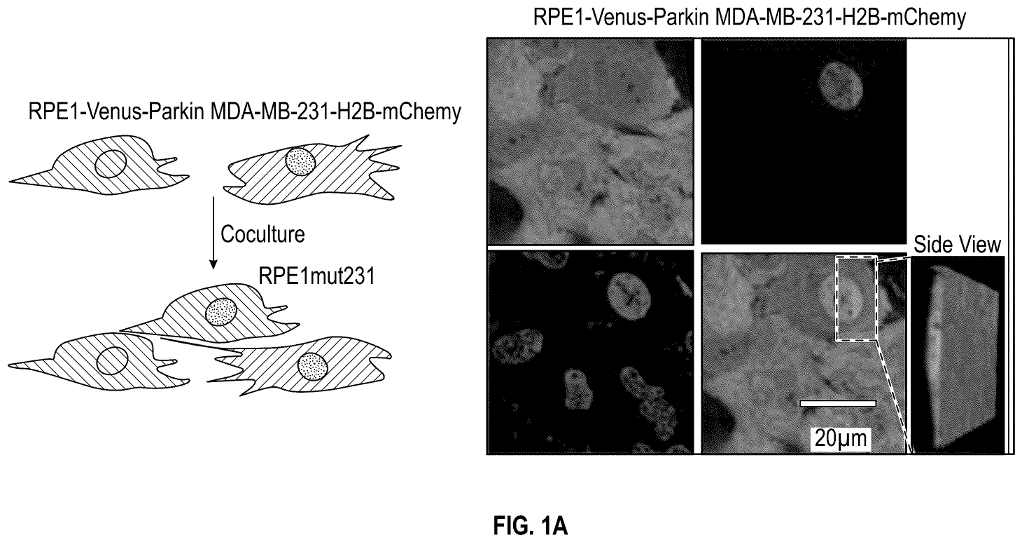

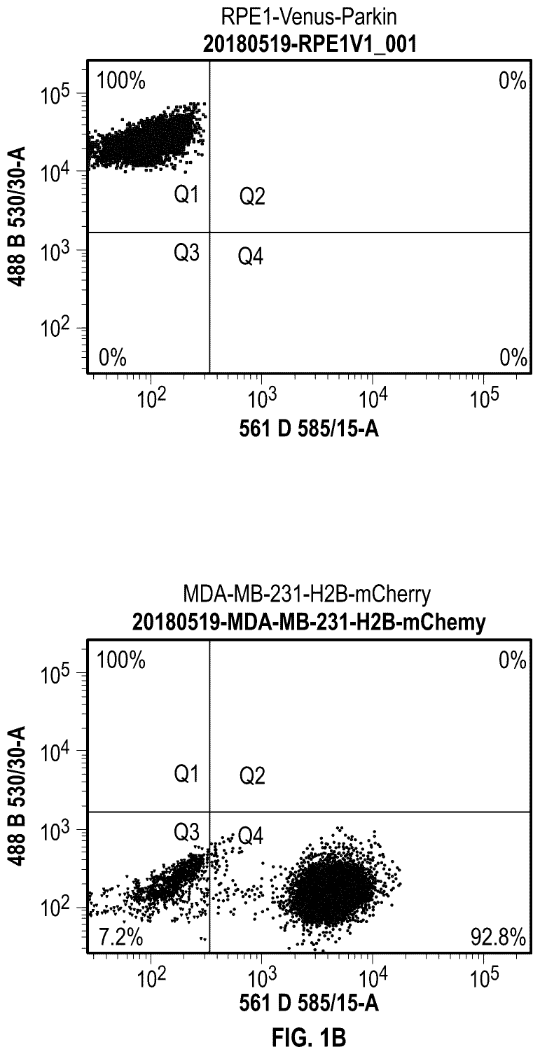

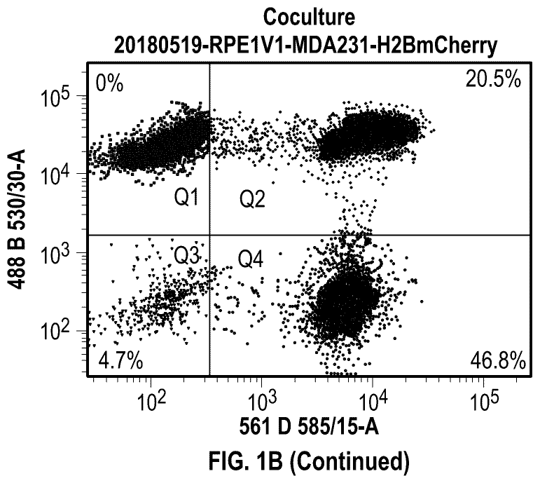

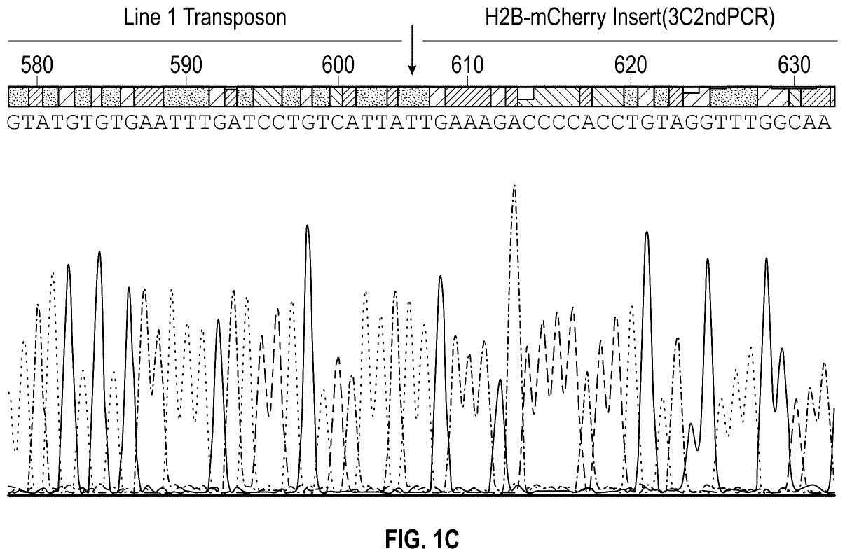

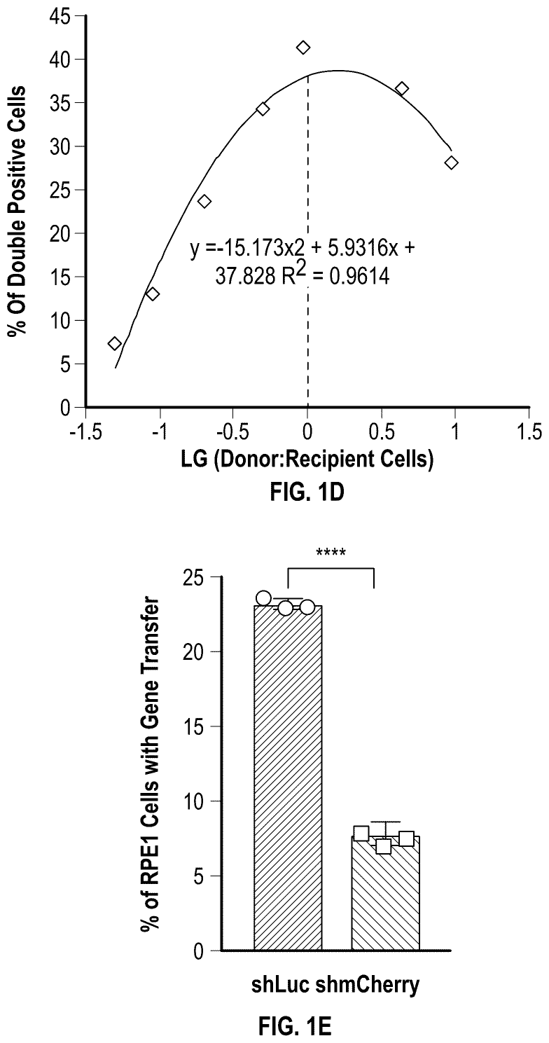

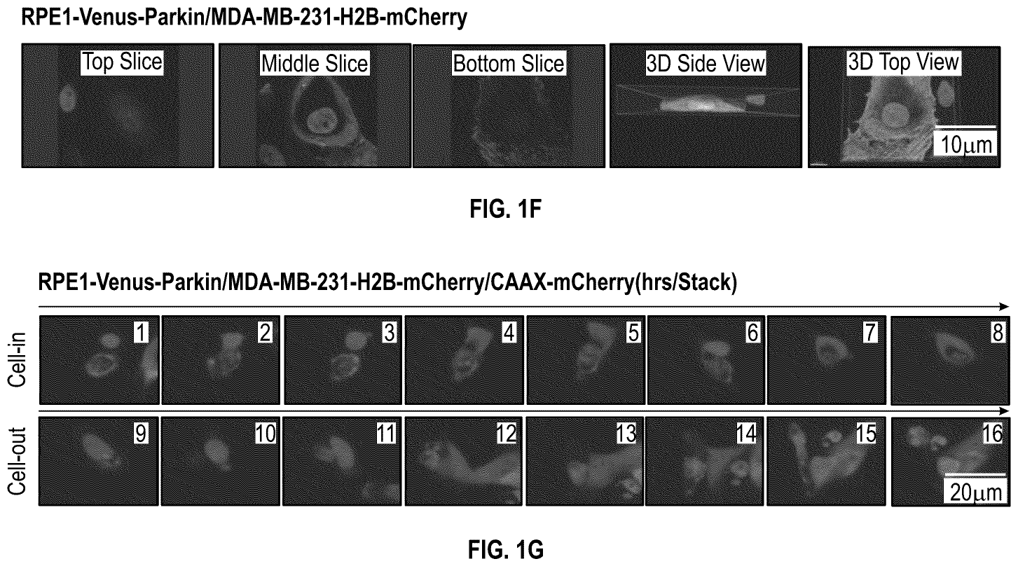

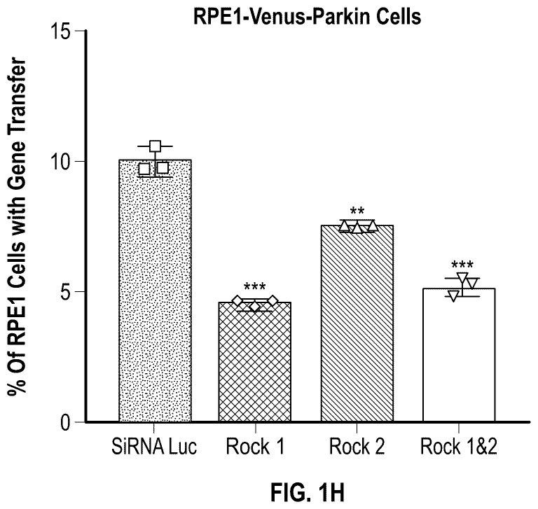

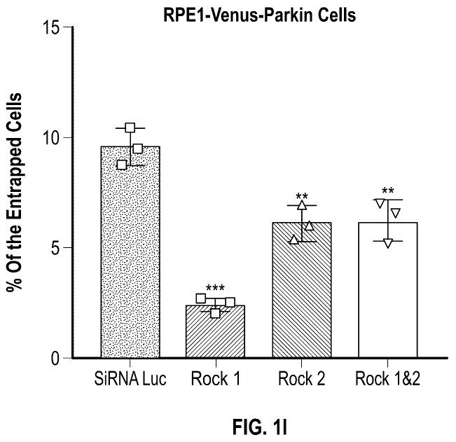

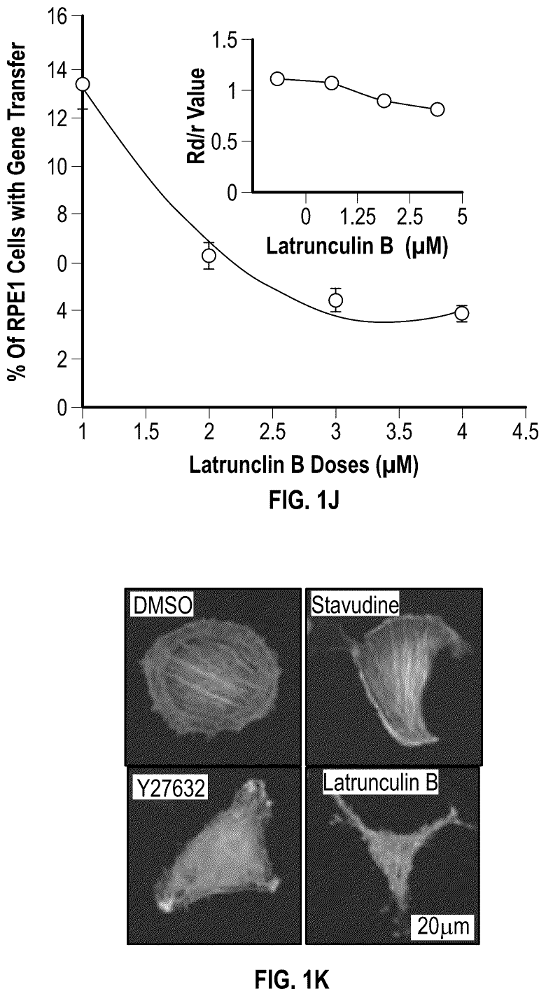

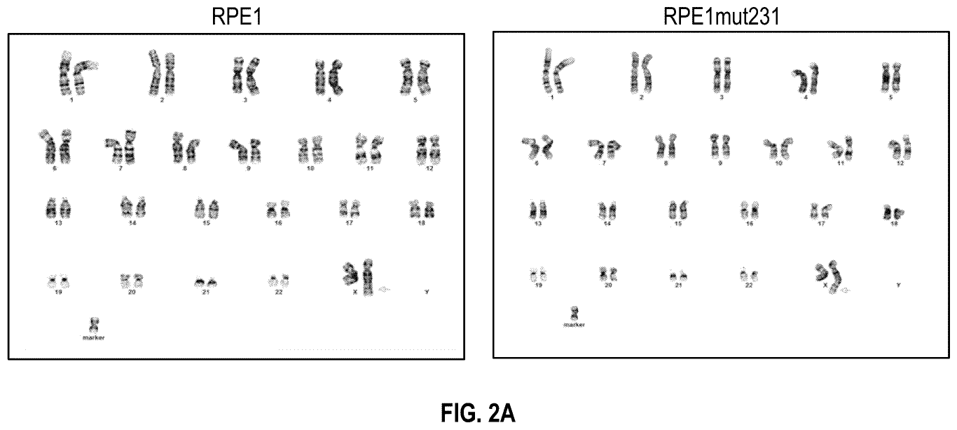

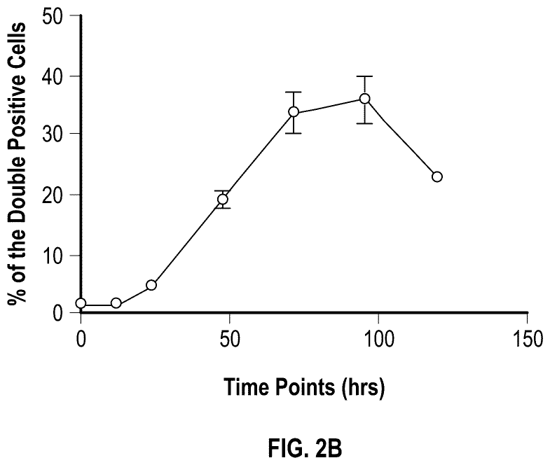

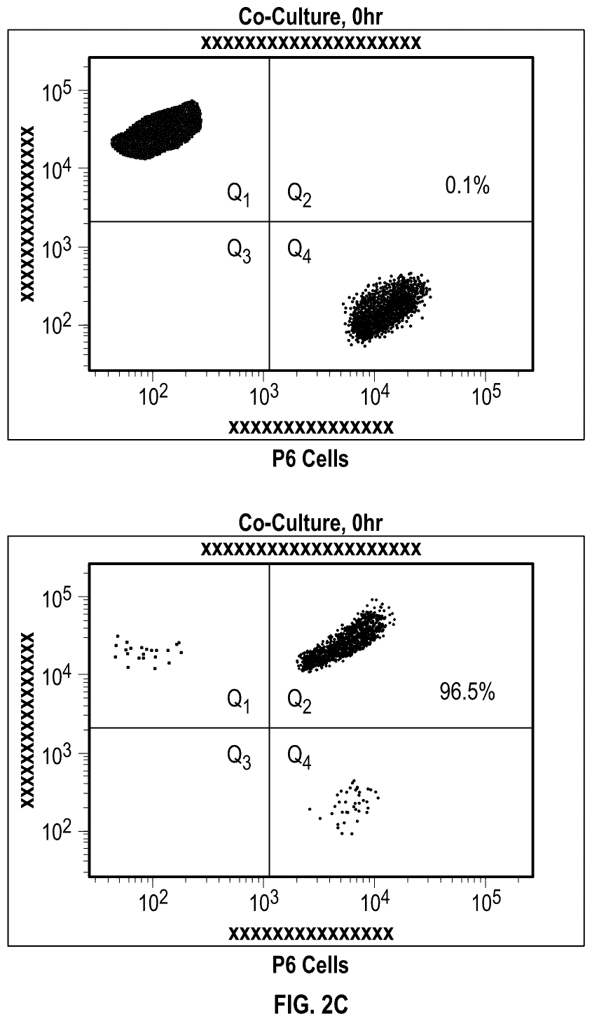

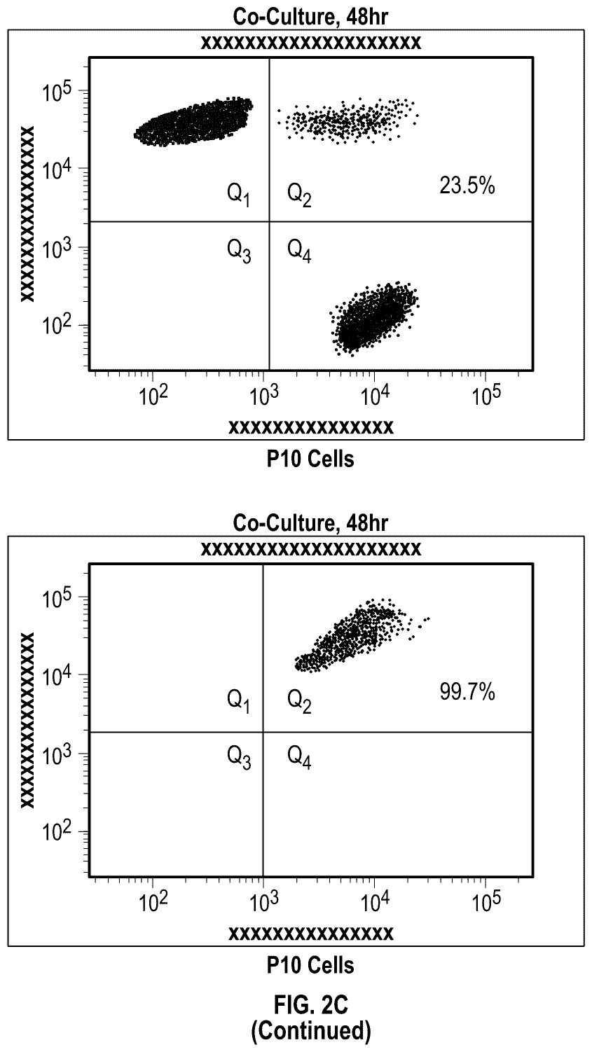

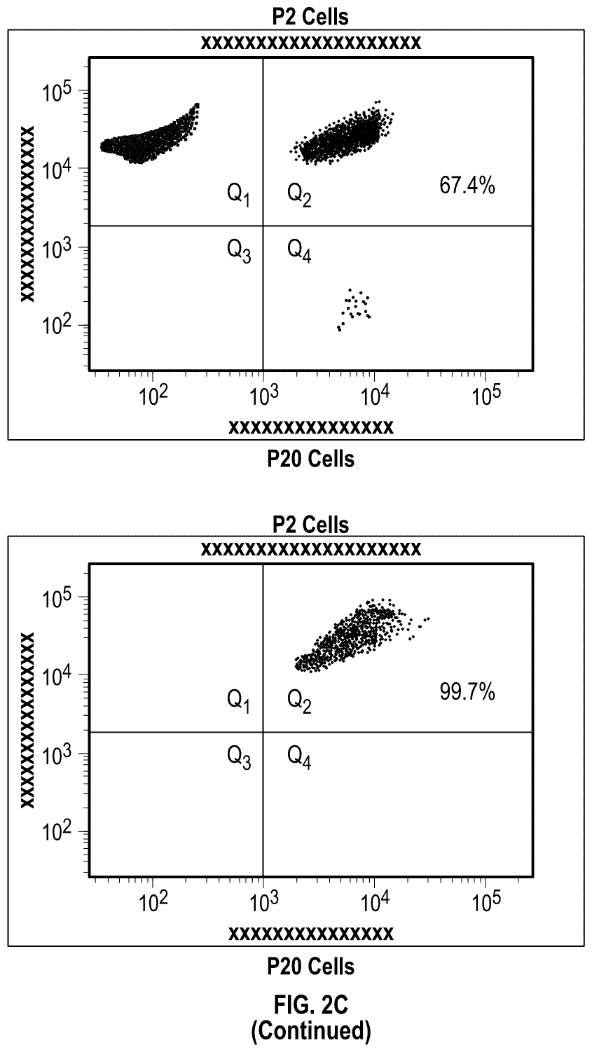

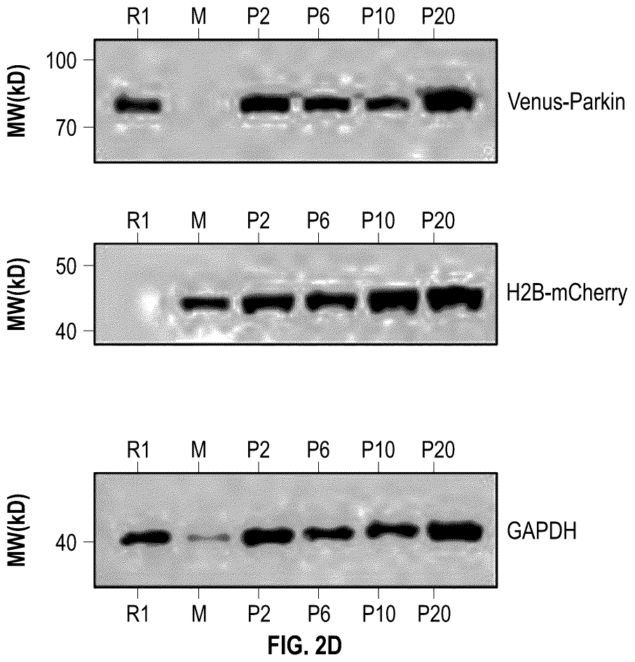

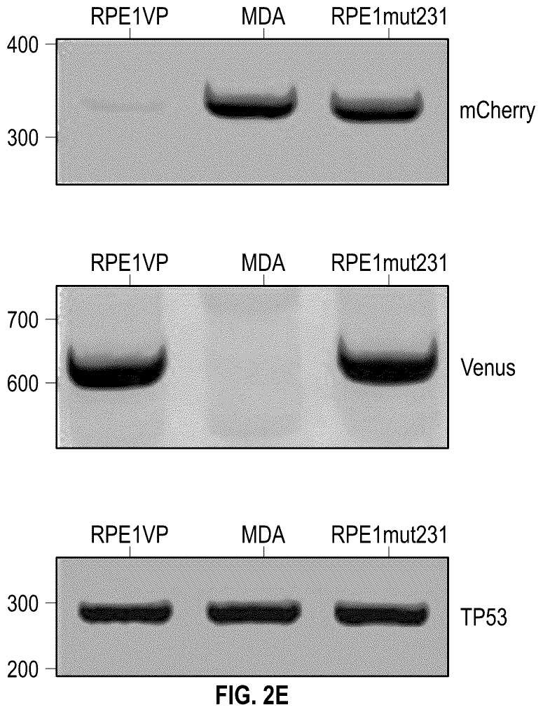

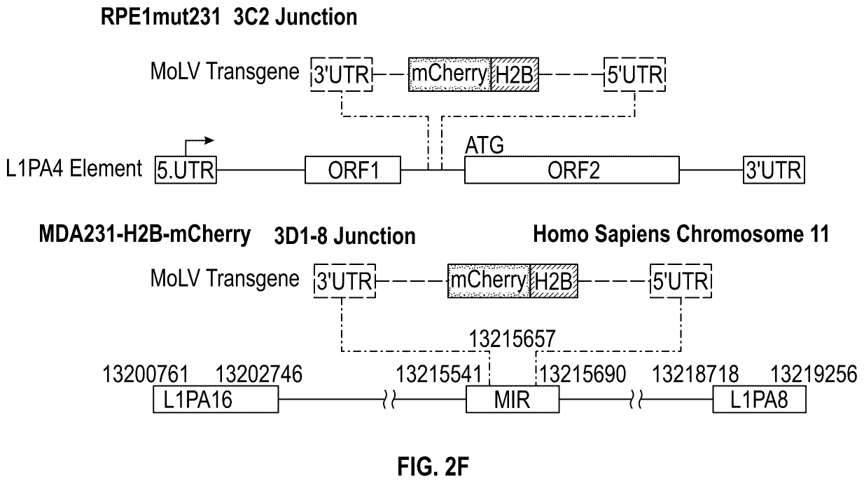

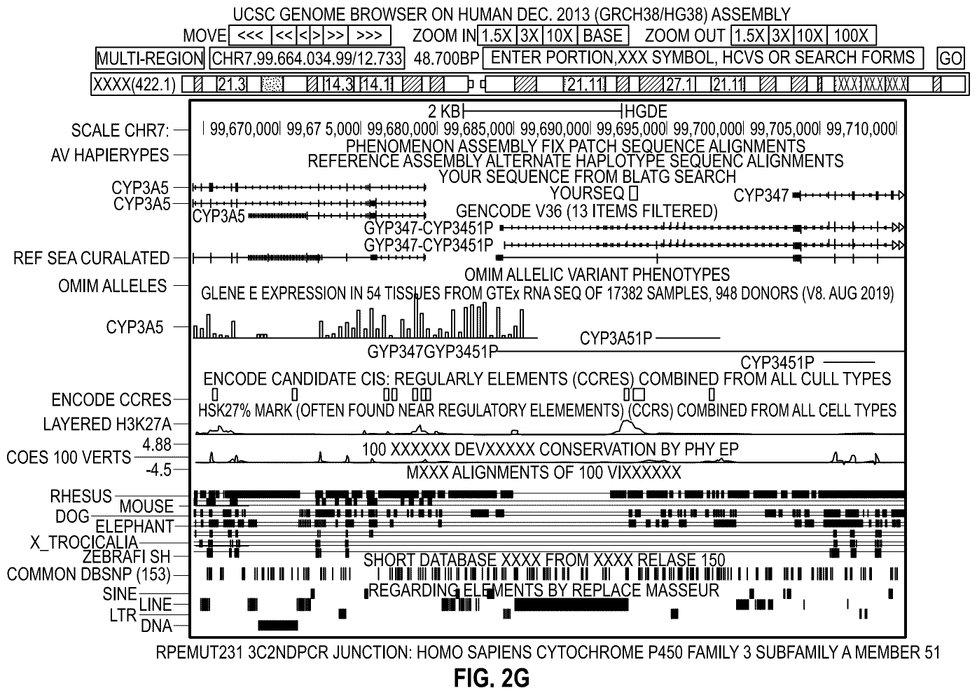

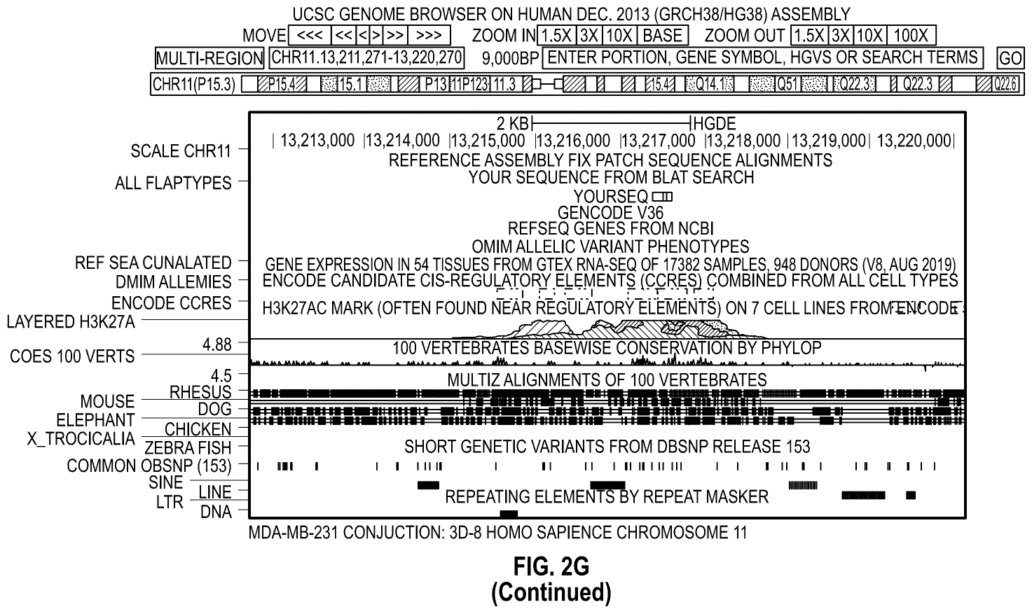

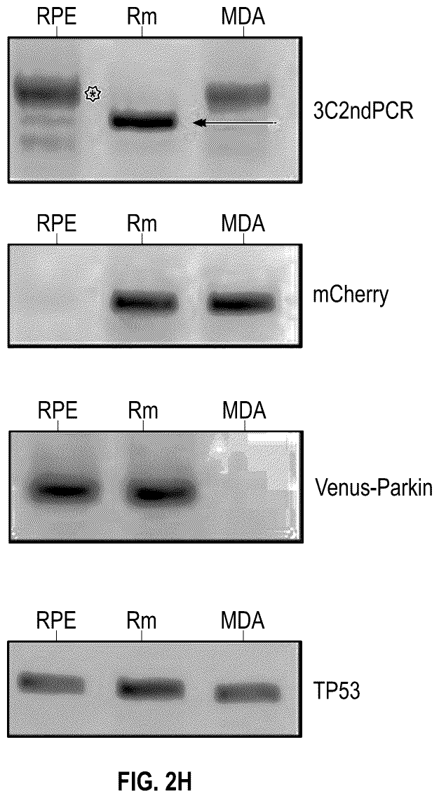

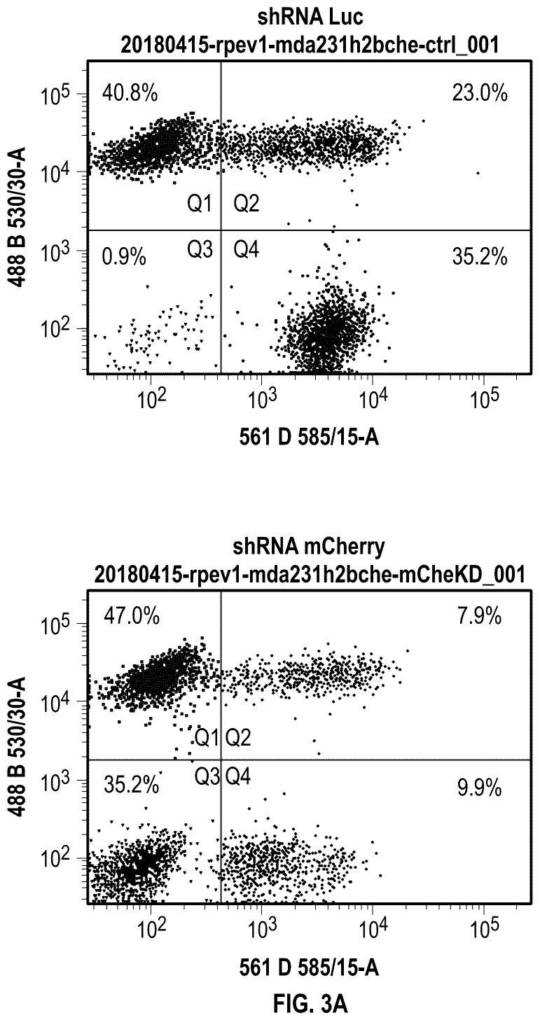

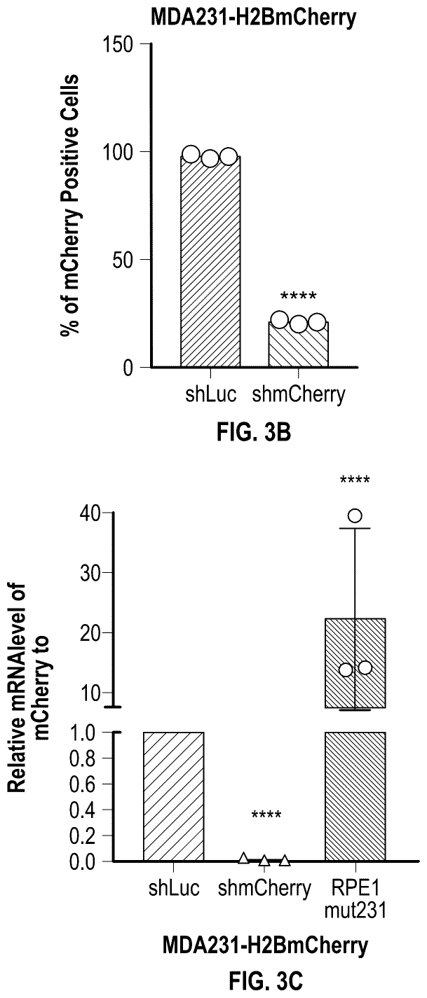

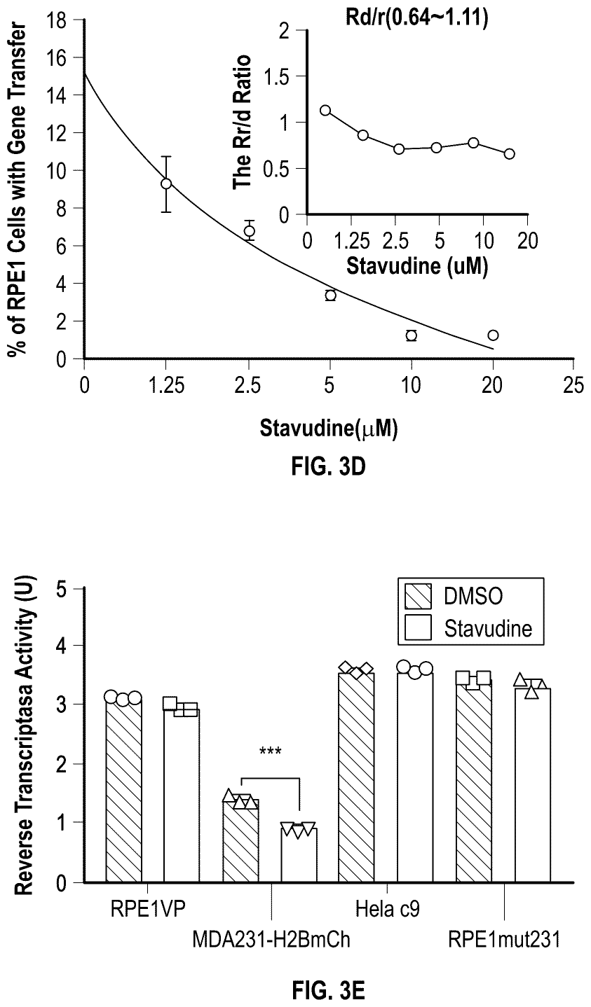

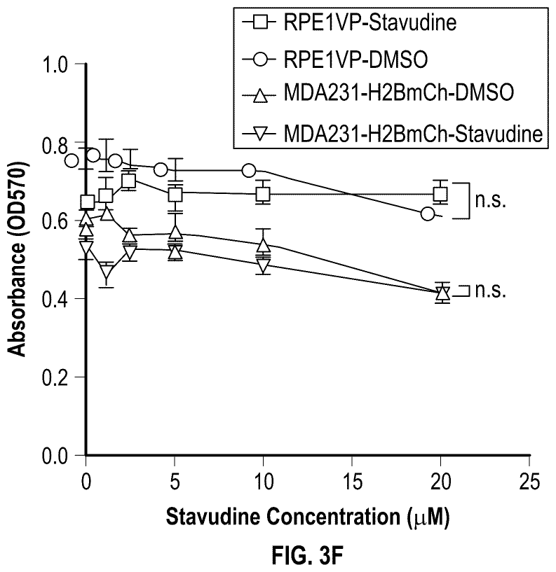

In an experiment to study the mitochondrial recruitment of Parkin, a critical regulator of mitophagy, the present inventors co-cultured RPE1, an immortalized non-transformed pigmented epithelium cell line stably expressing Venus-Parkin, and MDA-MB-231, a metastatic breast cancer cell line stably expressing H2B-mCherry. Unexpectedly, after 48 hours of co-culture, the present inventors observed that ˜20% of the mixed cell populations became double-positive for both Venus-Parkin and H2B-mCherry by both confocal microscopy and flow cytometry analysis (FIGS. 1a, b). The double-positive cells were isolated by cell sorting and further analyzed by karyotype analysis. All double-positive cells carry 46 chromosomes plus a small marker chromosome (n=10, Supplementary FIG. 2a), which is identical to the karyotype of the parental diploid RPE1 cells. The ratios of double-positive cells peak at 96 hs and decline at later time points, which may be a result of the higher rate of proliferation of non-transduced RPE1 cells, or reduced proliferation rate of parental MDA-MB-231 cells in the co-cultured environment, or reduced cell-cell interaction when the confluence of co-culture cells increases (Supplementary FIG. 2b).

Double positive cells (referred as RPE1mut231) from co-culture and cell sorting are stable as they can be perpetuated indefinitely (Supplementary FIGS. 2c, d). PCR analysis of the genomic DNA isolated from RPE1mut231 cells shows the H2B-mCherry DNA is present in these cells but not the parental RPE1 cells (Supplementary FIG. 2e), suggesting H2B-mCherry has been stably transferred from MDA-MB-231 to RPE1. To further confirm gene transfer, the present inventors performed retroviral integration site analysis using the GenomeWalker technology with both cell lines. This analysis conclusively identified at least one MoLV-H2B-mCherry site in the donor cell on chromosome 11 within an interspersed repetitive (MIR) element sandwiched by two partial Line-1 elements. This region contains several ENCODE candidate Cis-Regulatory Elements featuring high levels of H3K27Ac mark (Supplementary FIGS. 2f, g). In RPE1mut231 cells, one recovered site shows that the MoLV-H2B-mCherry transgene (3C2ndPCR) is integrated 3 bp upstream of the initiation codon (ATG) of ORF2 of a Line-1 element LIPA4 that resides in CYP3A51P pseudogene on chromosome 7. The integration sites for H2B-mCherry appear to be distinct in the donor and recipient cells as confirmed by sequencing and locus-specific PCR analysis (FIG. 1c and Supplementary FIGS. 2c, f-h). Although this analysis by no means can identify all possible integration sites of the H2B-mCherry gene in both cell lines or implicate which transgene is more likely on the move, identification of non-identical integration sites between donor and recipient cells supports the conclusion that intercellular gene transfer occurred upon co-culturing these two cell lines.

### Example 2: Optimization of Co-Culturing System

Next, the present inventors set out to optimize the co-culture system for achieving the highest efficiency of gene transfer. Specifically, the present inventors investigated whether different ratios of donor and recipient cells affect gene transfer and found that the occurrence of RPE1mut231 peaked around 40% when co-cultured donor and recipient cell lines mixed at a 1:1 ratio (FIG. 1d). Variability of the efficiency of gene transfer is statistically insignificant when the mixing ratio (Rd/r) of the donor to recipient cells stays in specific ranges (i.e., 0.8 vs. 0.9˜ 0.8 vs. 1.9) based on Post Hoc analysis. These analyses confirm that robust gene transfer occurs from MDA-MB-231 (donor) to RPE1 (recipient) cells.

### Example 3: Characterization of HGT Mediators

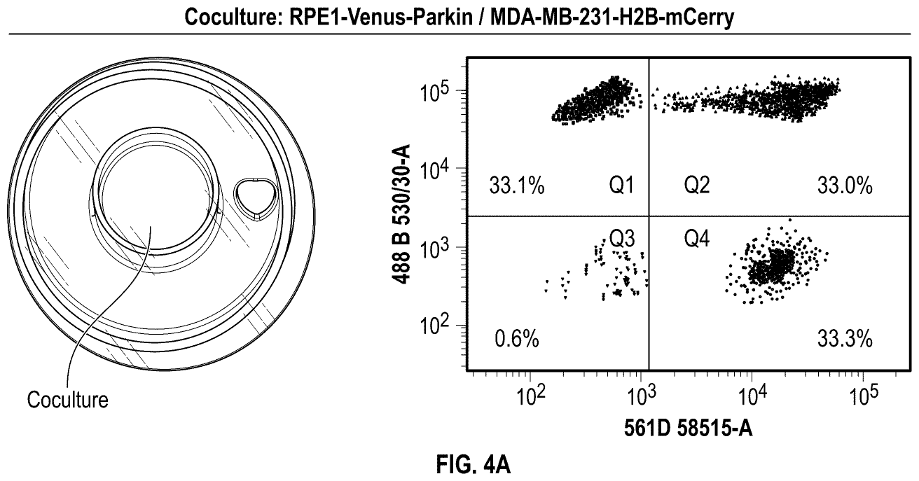

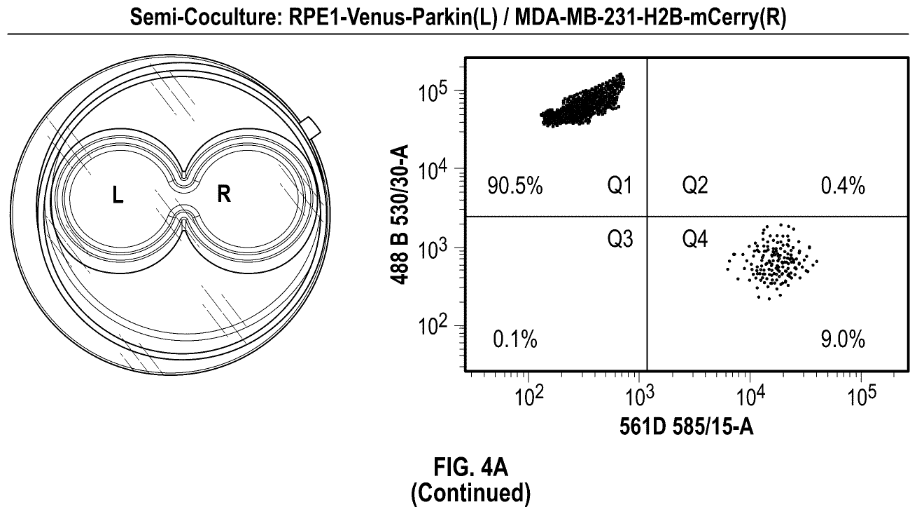

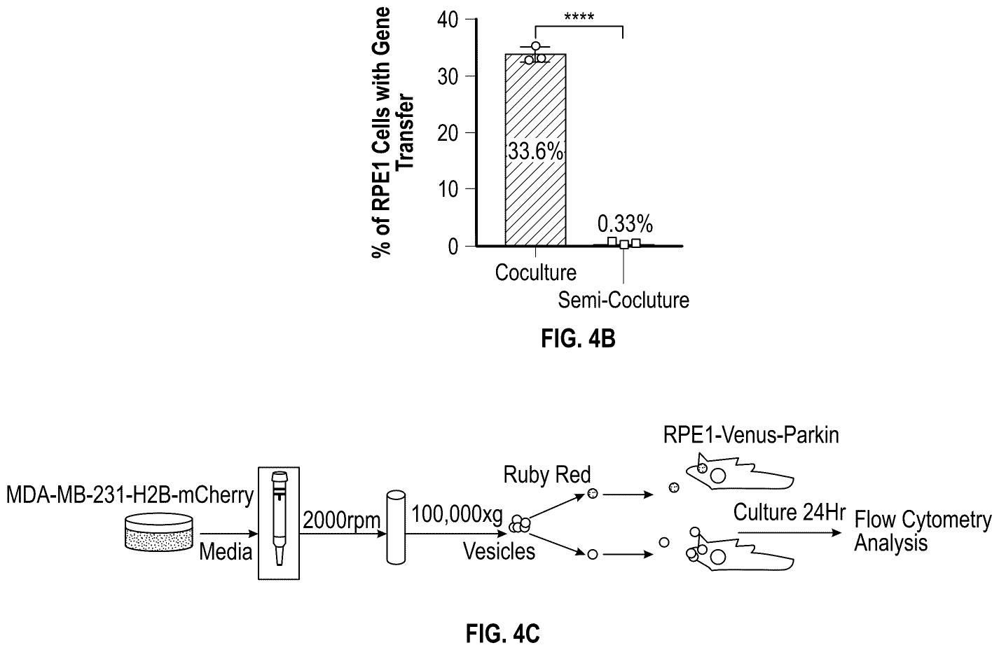

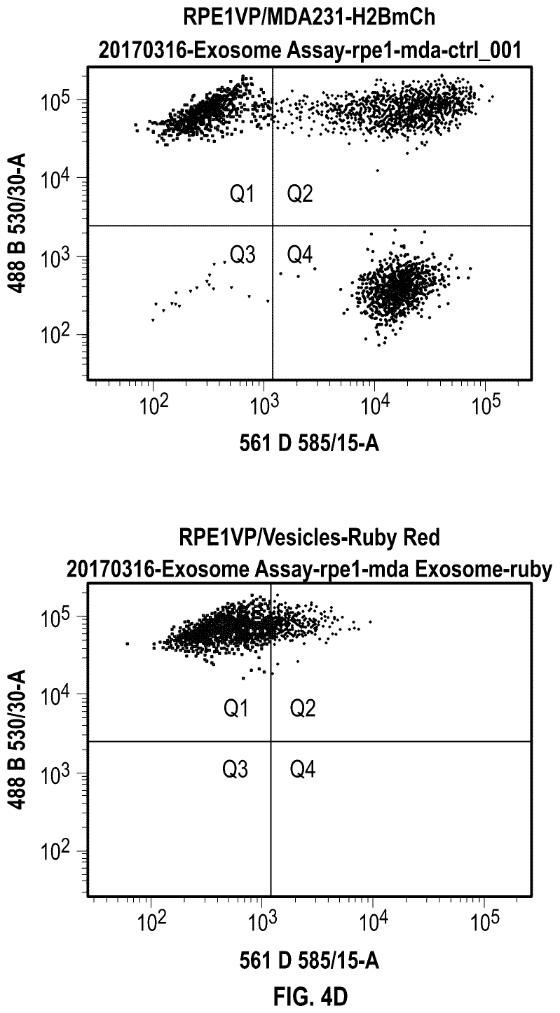

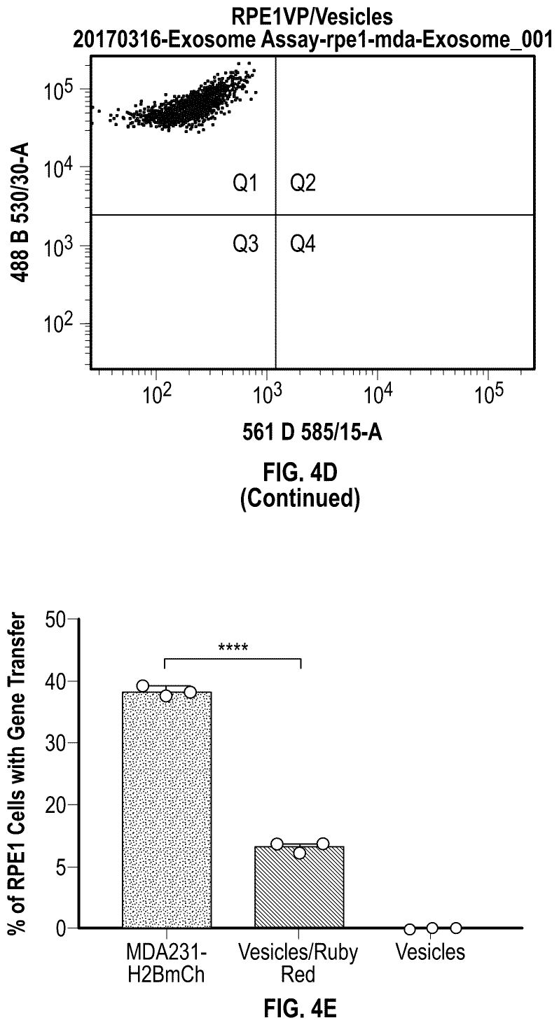

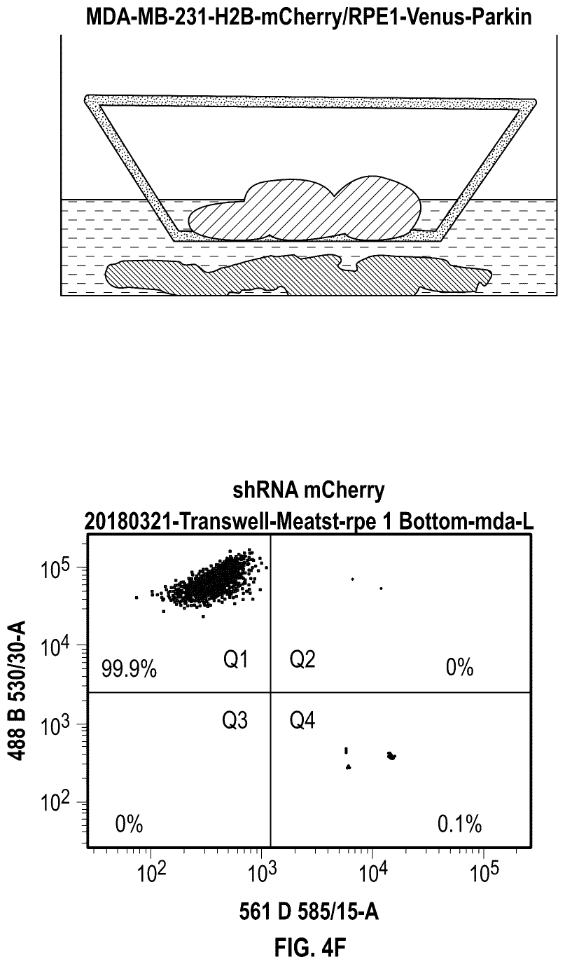

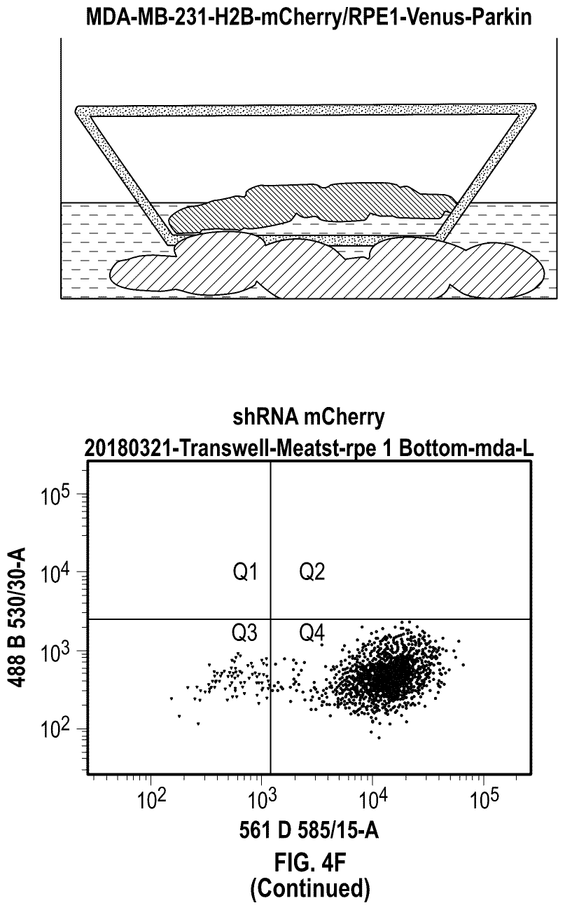

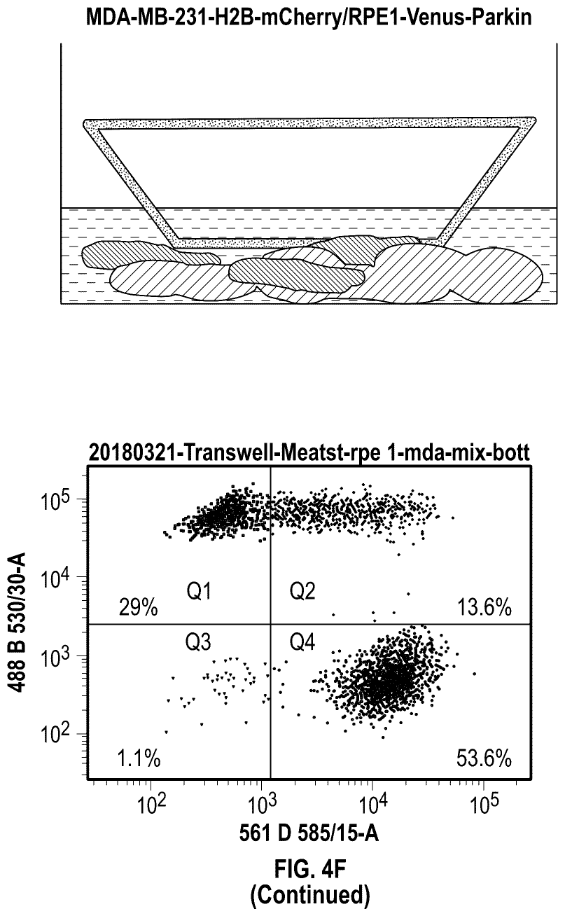

The present inventors further characterized the potential mediators of the novel HGT process. Neither the cultured supernatant from MDA-MB-231 (H2B-mCherry) cells nor extracellular vesicles obtained by ultracentrifugation of the donor cell supernatants (100,000×g), were able to transfer H2B-mCherry to RPE1 cells (Supplementary FIGS. 4a-c). Consistently, physical separation of donor and recipient cells reciprocally by the Transwell insert inhibited any detectable transfer by flow cytometry analysis (Supplementary FIG. 4f). These results are inconsistent with a cell-free transfer of genetic information via diffusible nucleoprotein complex, viruses, or extracellular vesicles, suggesting that cell-cell contact is required to achieve intercellular gene transfer.

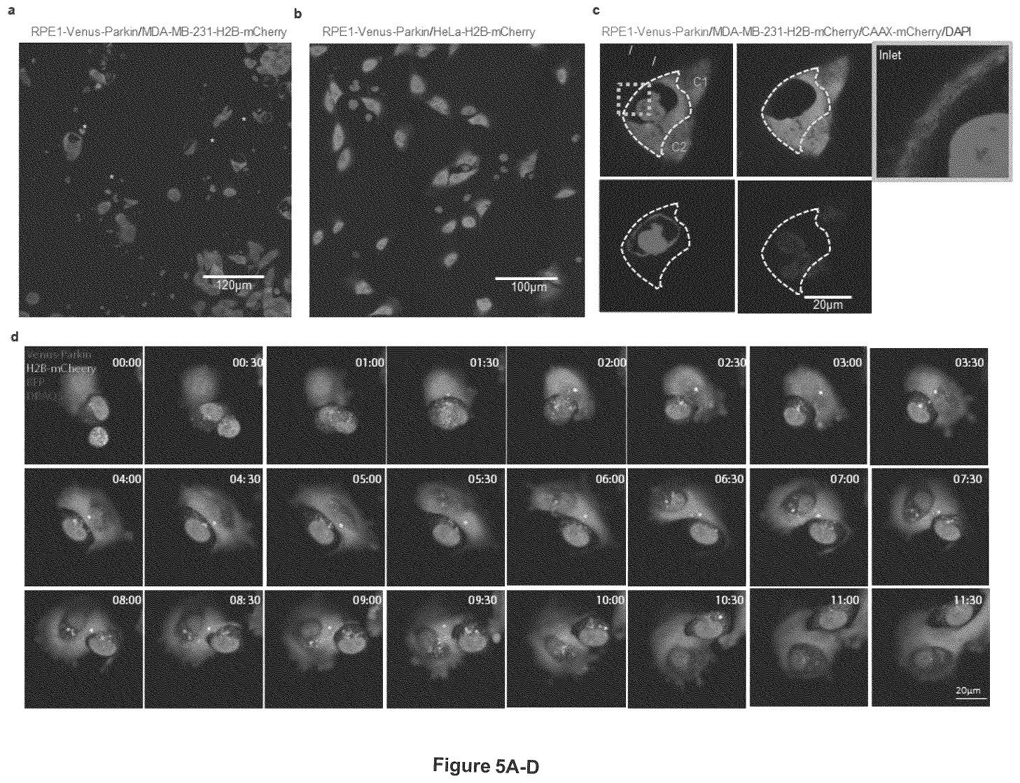

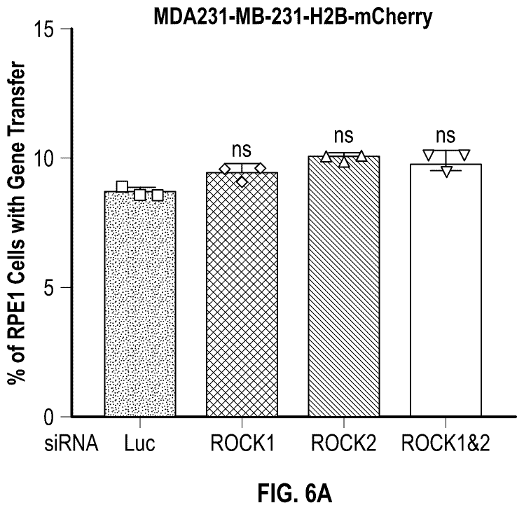

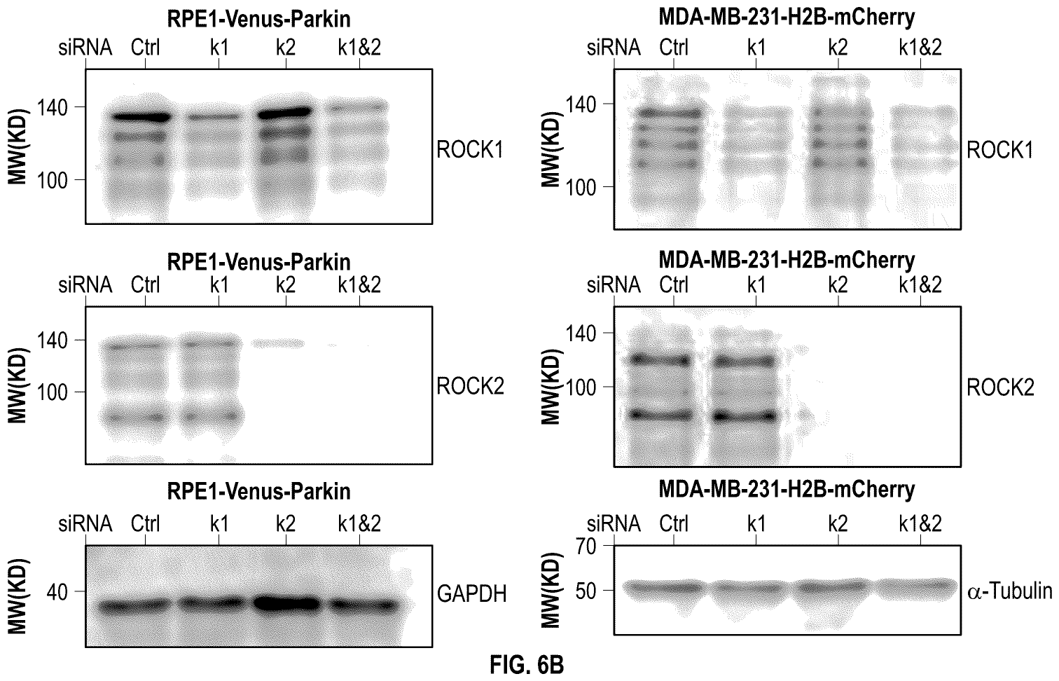

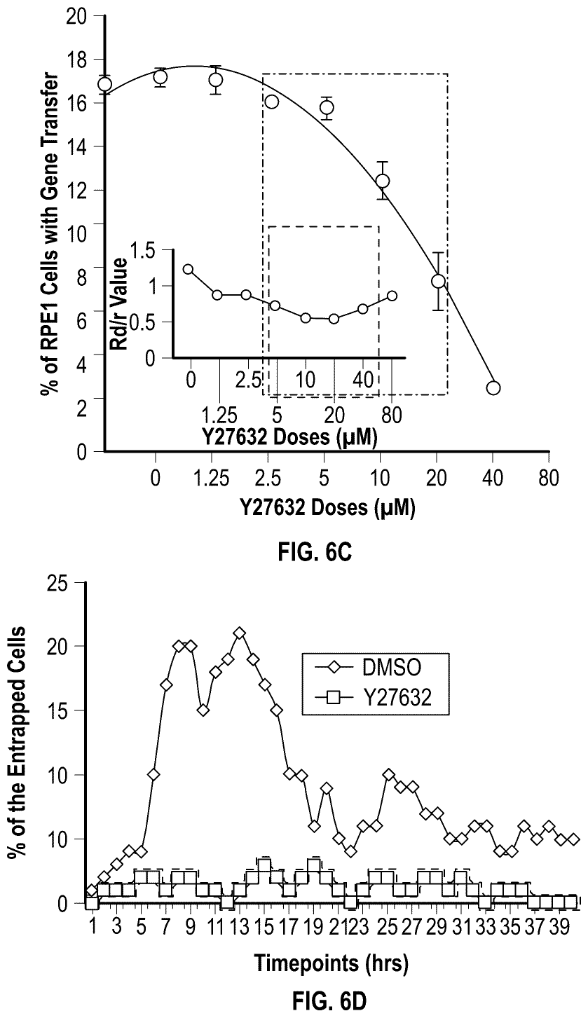

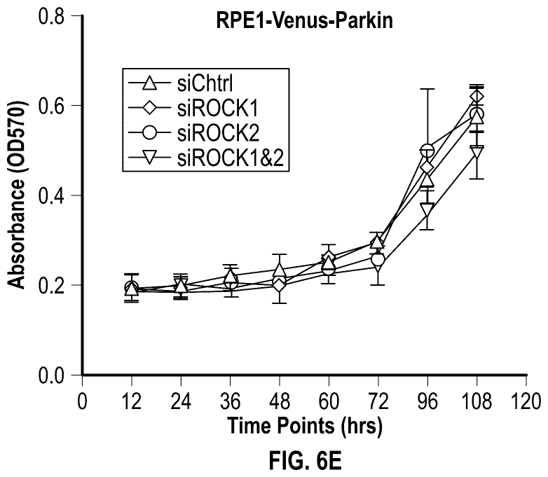

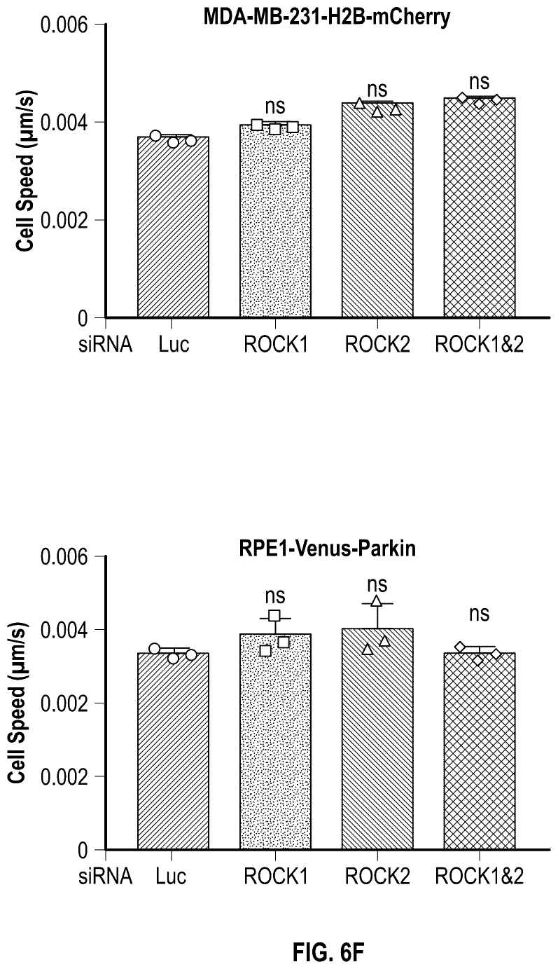

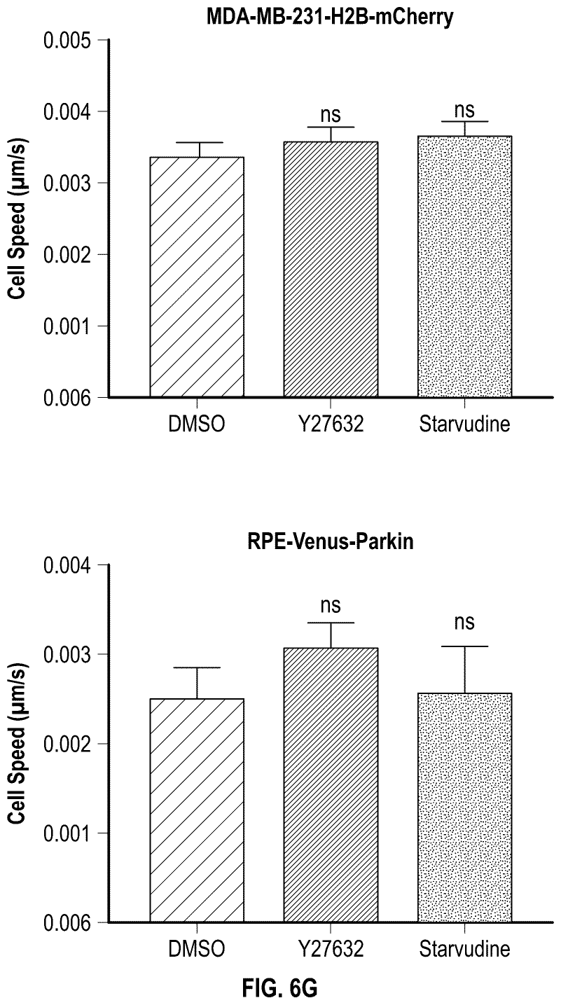

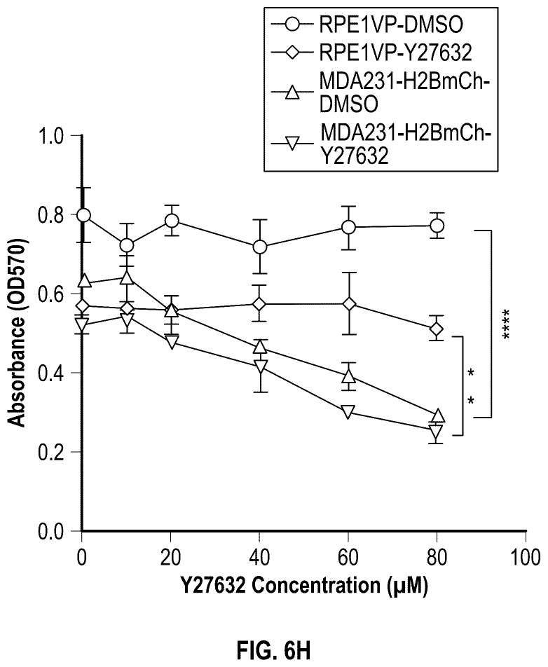

To visualize the dynamics of cell interactions and gene expression during the transfer, characterized the potential mediators of this process performed high content live-cell imaging of the interaction between the donor and recipient cells for a period of 11 to 24 hrs. The most notable feature upon the co-culturing of donor and recipient cells was the formation of the “cell-in-cell” like mosaic structure. The donor MDA-MB-231 (H2B-mCherry) cells were found trapped inside the recipient RPE1 (Venus-Parkin) cells frequently while maintaining their cellular boundaries (FIGS. 1f, g, Supplementary FIGS. 5a, c, d, and Supplementary Movie 1 and 2, incorporated herein by reference). The detention of donor cells by recipient cells is a transient and dynamic process. The percentage of cell mosaic structures increased significantly from 7 to 15 hours and decreased slowly after 19 hours, while the signal intensity of H2B-mCherry in the recipient cells' nuclei steadily increased over time (FIG. 1g and Supplementary FIGS. 2d, 6d). The donor and recipient cells' engagement in the cell mosaic state is reversible as the resolution of this state often occurs within hours. As high as 20% of donor cells entered the state of entrapment during the time course of imaging (Supplementary FIG. 6d), which closely matches the efficiency of gene transfer as determined by flow cytometry analysis (FIG. 1b). The entrapment of the donor cells to form the cell mosaic state is cell type-specific since this process was not observed between RPE1 (Venus-Parkin) and HeLa (H2B-mCherry) (Supplementary FIG. 5b and Supplementary movie 3). Thus, these observations suggest that cell entrapment is a cell type-specific process.

### Example 4: Characterization of Cell Mosaic Structure Formation Mediated by ROCK Kinase 1/2

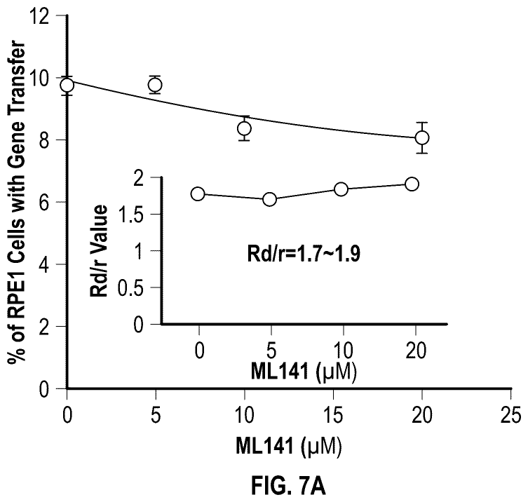

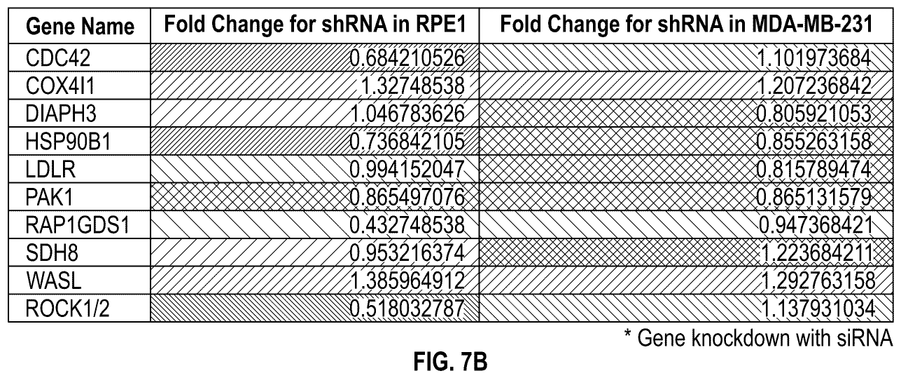

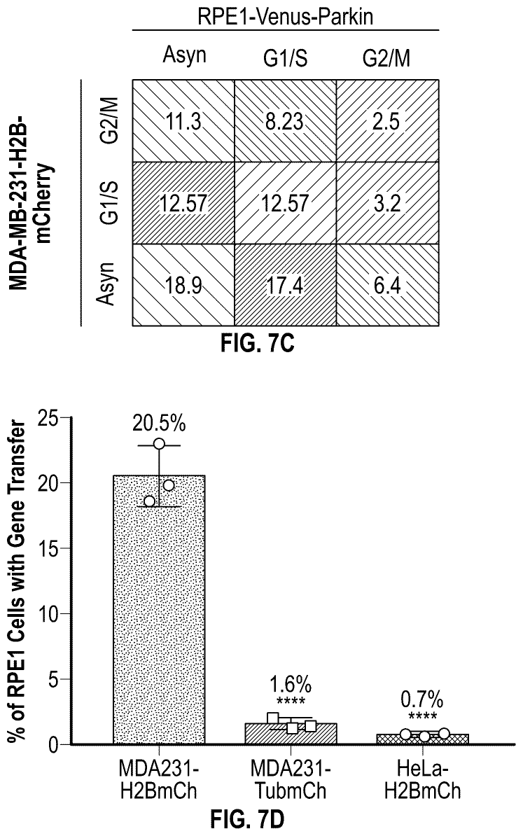

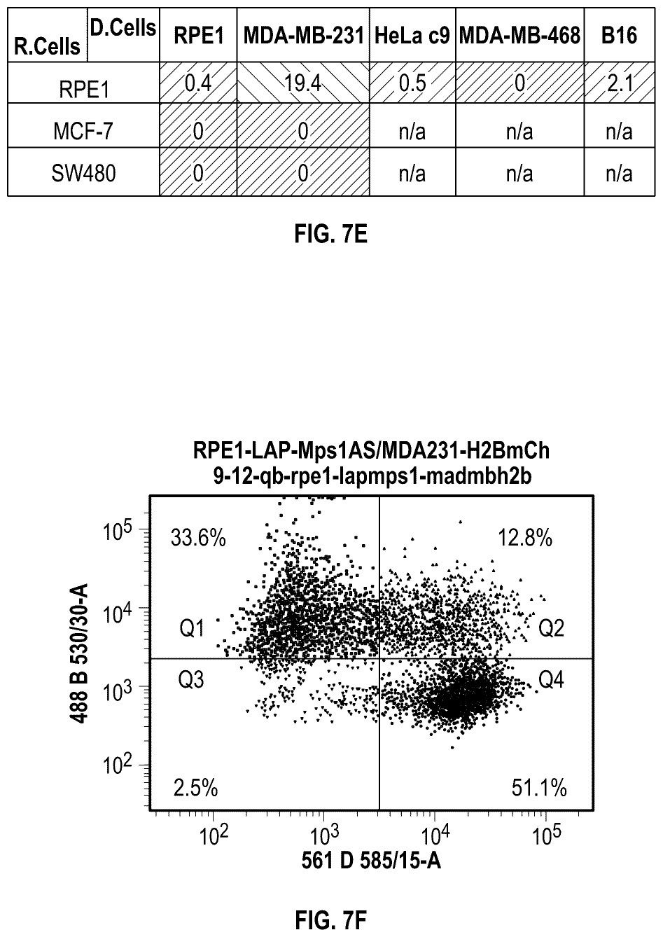

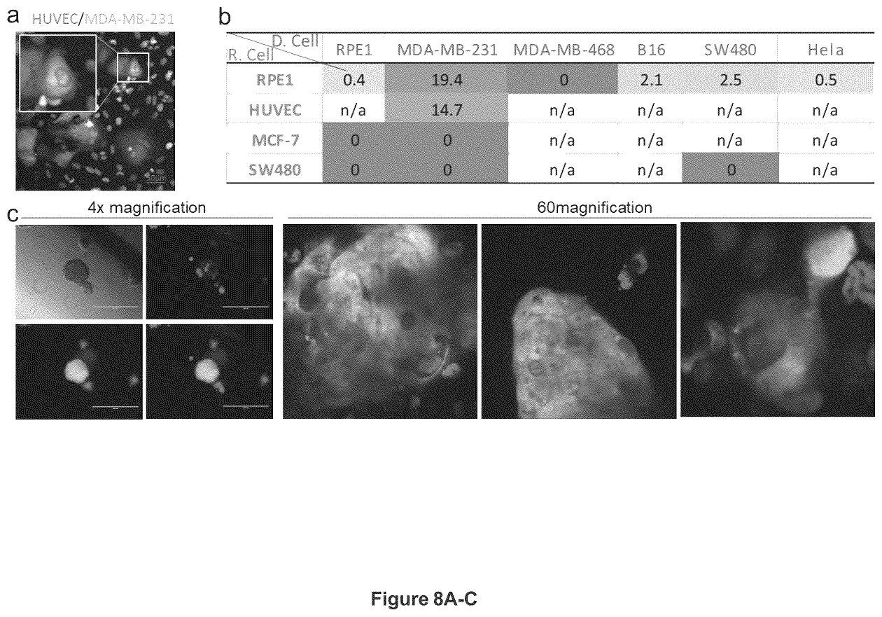

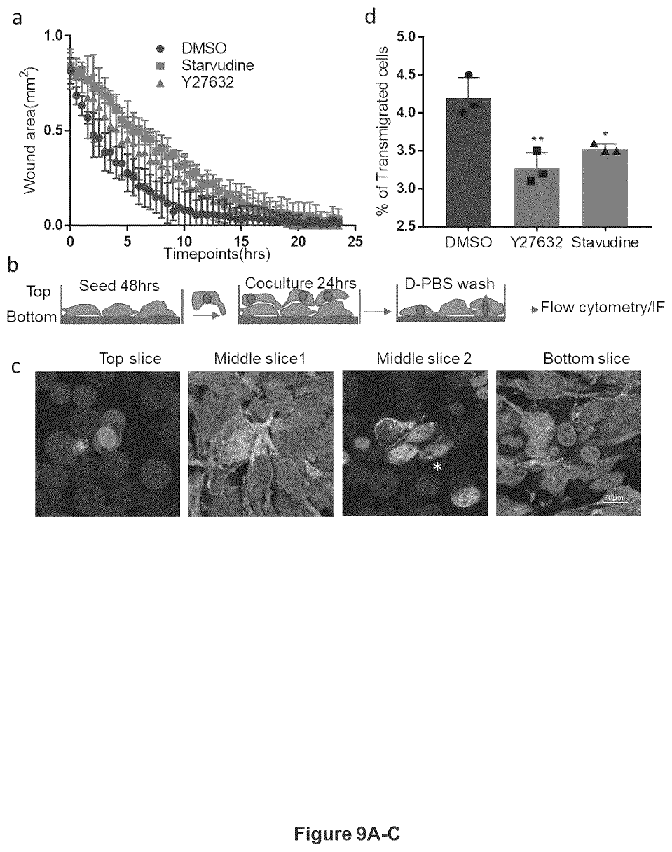

The formation of the cell mosaic structure may require coordinated intercellular communications and likely specific receptor/ligand interactions. To identify the cellular effectors that regulate intercellular gene transfer, the present inventors performed a small set mRNA knockdown screen with genes known to be involved in entosis and uncovered ROCK kinase 1/2 as strong hits for this process (Supplementary FIG. 7b). Depletion of ROCK1 and ROCK2 using siRNA in recipient cells can abrogate both gene transfer and cell entrapment (FIGS. 1h, 1i). In contrast, depleting ROCK1 and ROCK2 in donor cells has no major effect on gene transfer (Supplementary FIGS. 6a, b). The effect of ROCKs perturbation by RNA depletion was phenocopied by the treatment of ROCK kinase inhibitor Y27632 (Supplementary FIGS. 6c, d). The perturbation of gene transfer is unlikely due to inhibition of cell proliferation and motility as both are barely affected by ROCK1/2 inactivation in either donor or recipient cells (Supplementary FIGS. 6e-h). Since ROCK kinases are involved in actin cytoskeleton organization, the present inventors further tested whether Latrunculin B, an actin polymerization inhibitor, can block this process. As expected, incidence of gene transfer was significantly decreased, along with F-actin levels in the presence of Latrunculin B (FIGS. 1j, 1k). These results underscore the importance of actin dynamics as a key regulator of cell entrapment and intercellular gene transfer.

### Example 5: Novel Cell Entrapment is Differentiated from Entosis

The process described here, at first glance, shares some similarities to entosis. For example, both require ROCK kinases activity. However, they are fundamentally different in several aspects. First, E-cadherin is required for entosis. MDA-MB-231 cells do not express E128 Cadherin and are known to be incompetent to undergo entosis; yet these cells can pair with RPE1 to perform gene transfer. Secondly, depletion of CDC42 kinase can trigger mitotic entosis in adherent cells. However, depletion of CDC42 in donor or recipient cells has the opposite effect on gene transfer: gene transfer decreased when CDC42 was depleted in recipient cells or inhibited by ML141 inhibitor in co-cultured cells, but increased when it was depleted only in donor cells (Supplementary FIGS. 7a, b). This observation indicates that induction of mitosis in donor cells, rather than recipient cells, can promote gene transfer (Supplementary FIG. 7c). Finally, entosis is a rare event that involves whole chromosome gains or losses due to cytokinesis failure. Gene transfer by cell entrapment is highly efficient without significant changes in karyotypes (Supplementary FIG. 2a). Robust intercellular gene transfer is probably an intrinsic property of MDA-MB-231 as the H2B-mCherry gene in other cell lines (i.e., HeLa) can barely be transferred to RPE1-Venus-Parkin cells (Supplementary FIGS. 7d, e). A similar transfer frequency of H2B-mCherry can be achieved with independently generated MDA-MB-231 stable cells. However, -Tubulin-mCherry in MDA-MB-231 transferred poorly to RPE1-Venus-Parkin cells (<2%) (Supplementary FIGS. 7d, c). The intercellular gene transfer between MDA-MB-231 (H2B-mCherry) and RPE1-Venus-Parkin is also independent of Venus-Parkin as RPE1 cells stably expressing GFP-Mps1 are as competent as RPE1 (Venus-Parkin) to serve as the recipient cells (Supplementary FIG. 7f). This result suggests that the efficiency of this process is gene-specific in donor cells, but not in recipient cells. Future studies should pinpoint the genetic requirements for the robust transfer between RPE1 and MDA-MB-231 cells and whether other endogenous cellular genes can be transferred by this mechanism. Heterotypic or homotypic cell engulfment has been observed in certain tumor tissues. It will be interesting to determine whether some of these interactions are reversible cell entrapment to fuel genetic heterogeneity and tumor evolution via HGT. The finding that MDA-MB-231 cells can dive into RPE1 cells via a transient entrapment process raises an interesting possibility that this could be a mechanism for tumor cells to breach the epithelial barriers and metastasize to distant organs.

### Example 6: Identification of Small-Molecule Inhibitors of Tangocytosis

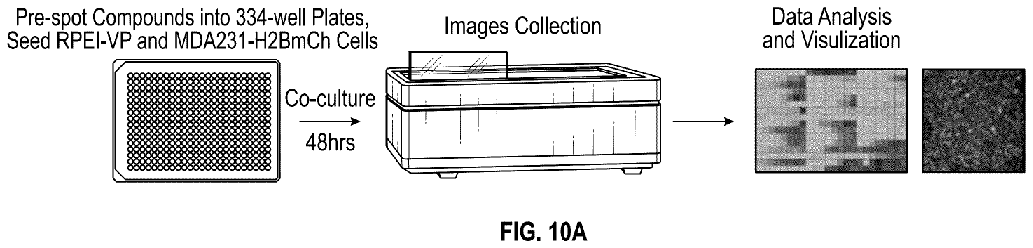

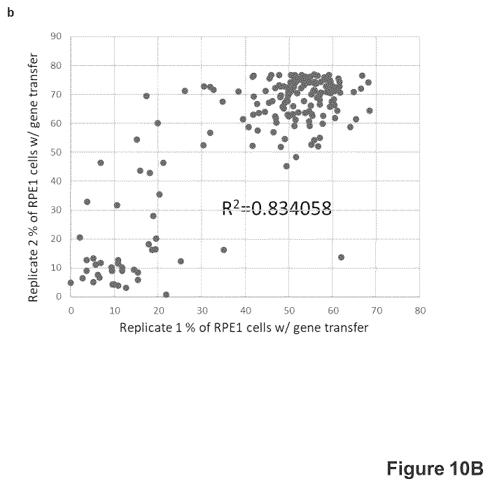

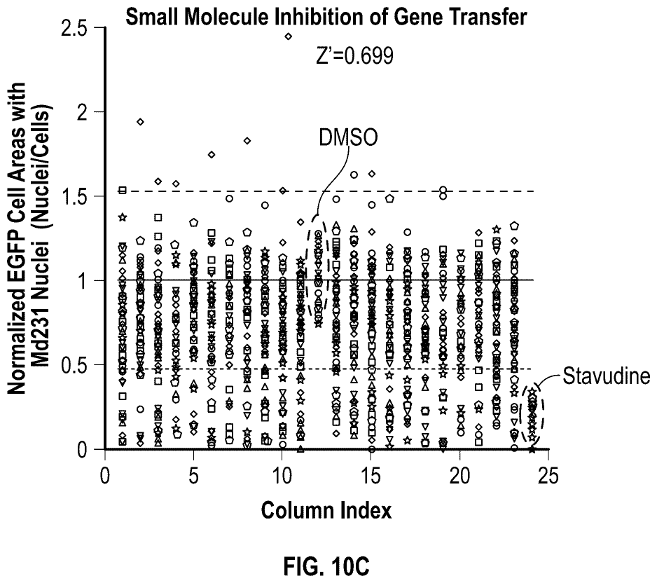

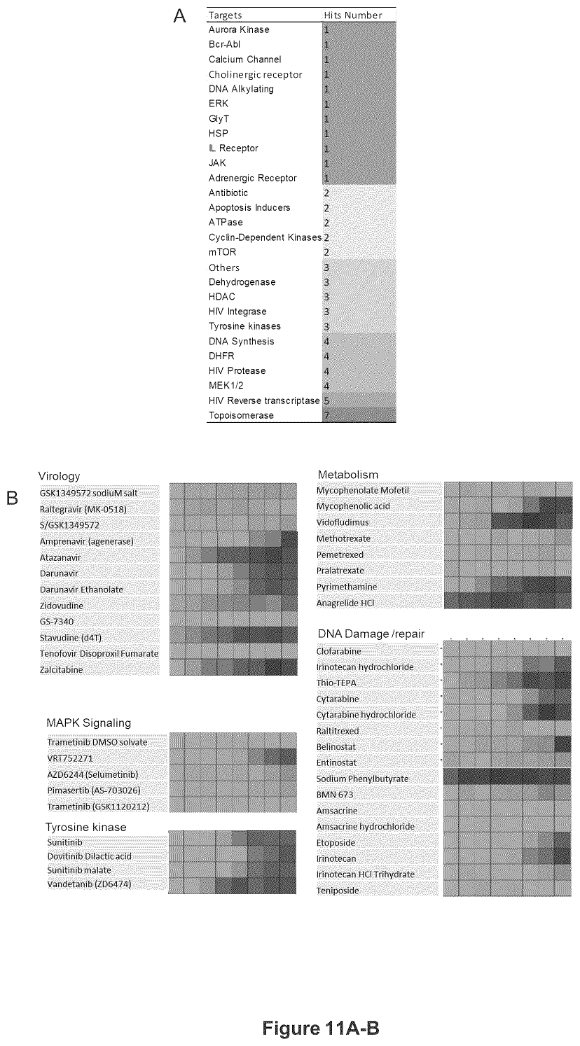

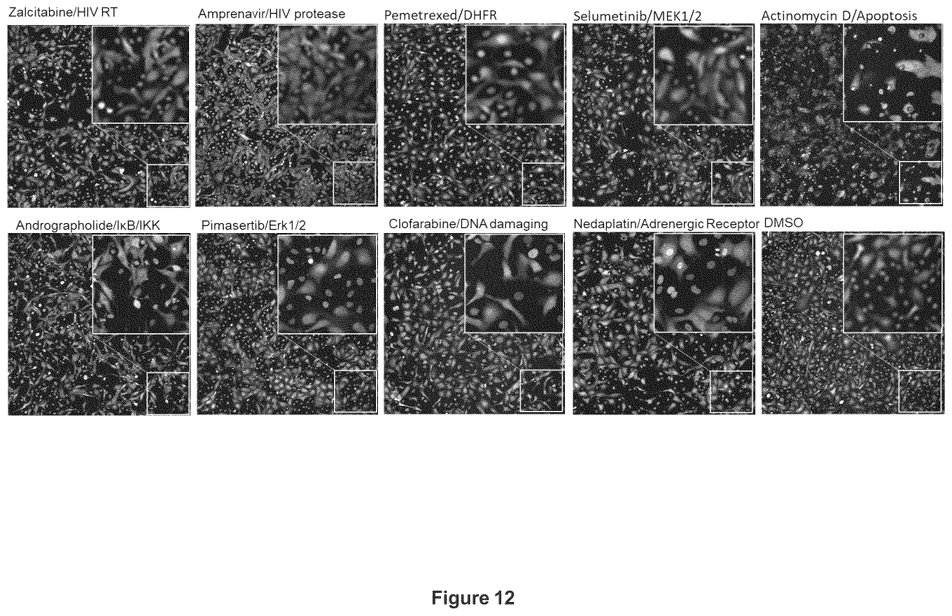

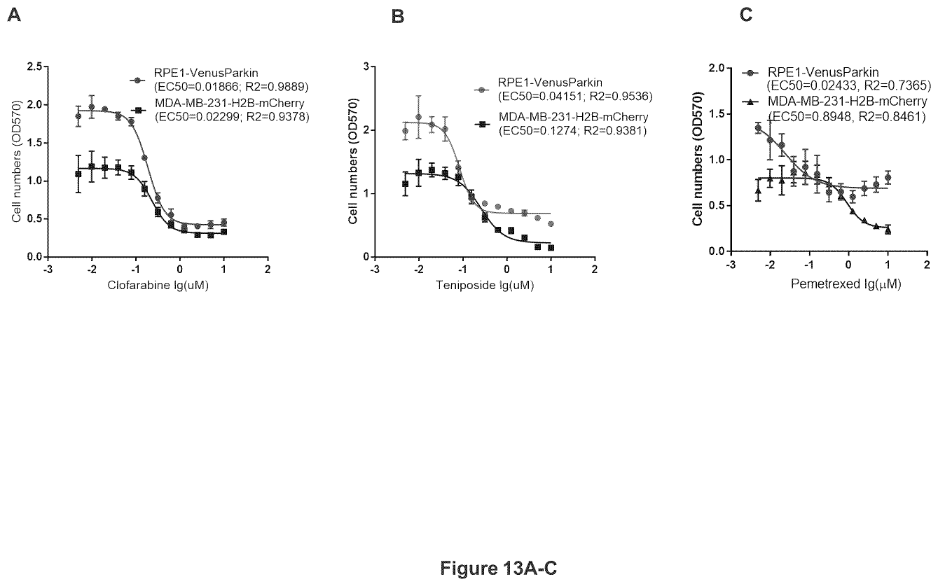

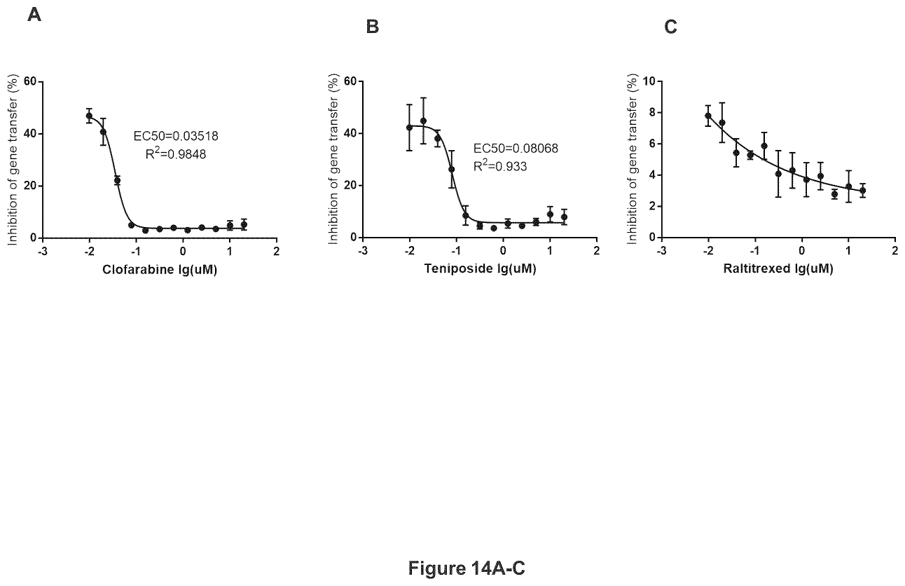

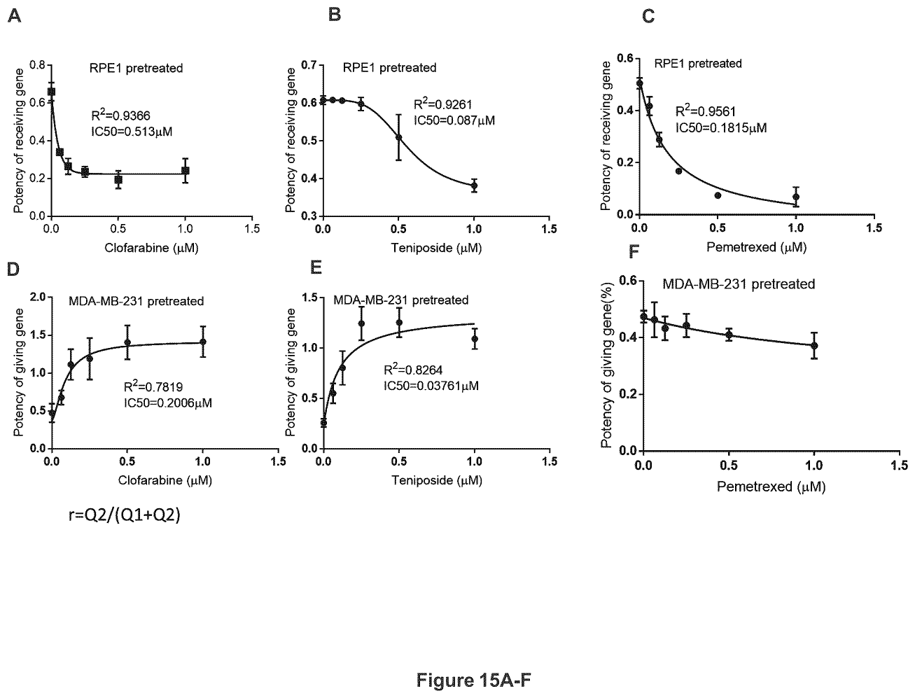

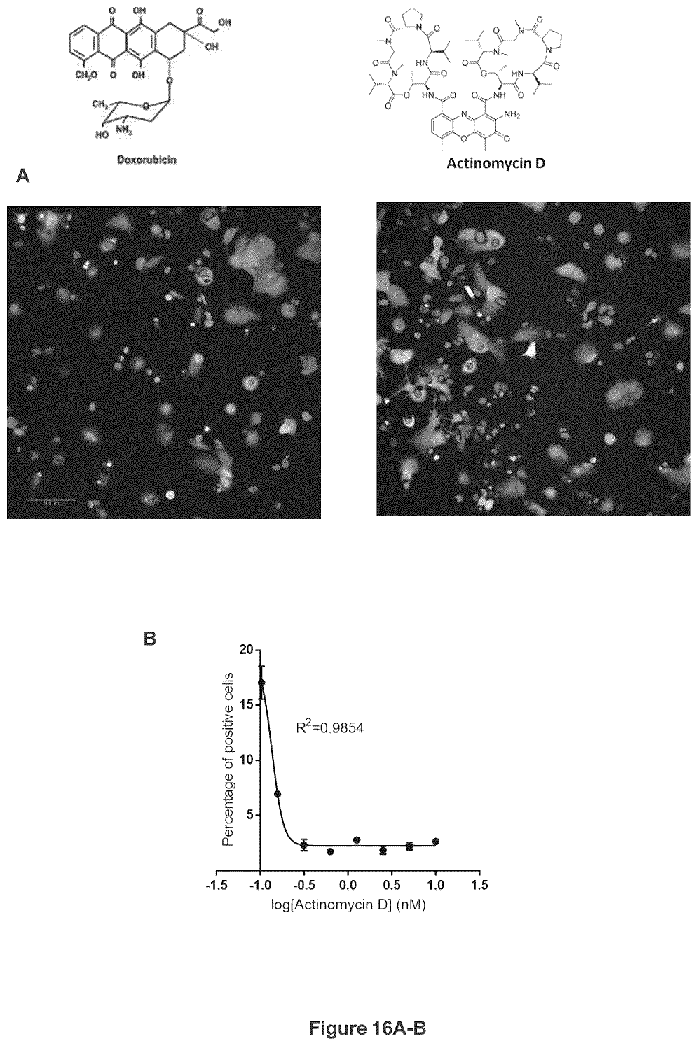

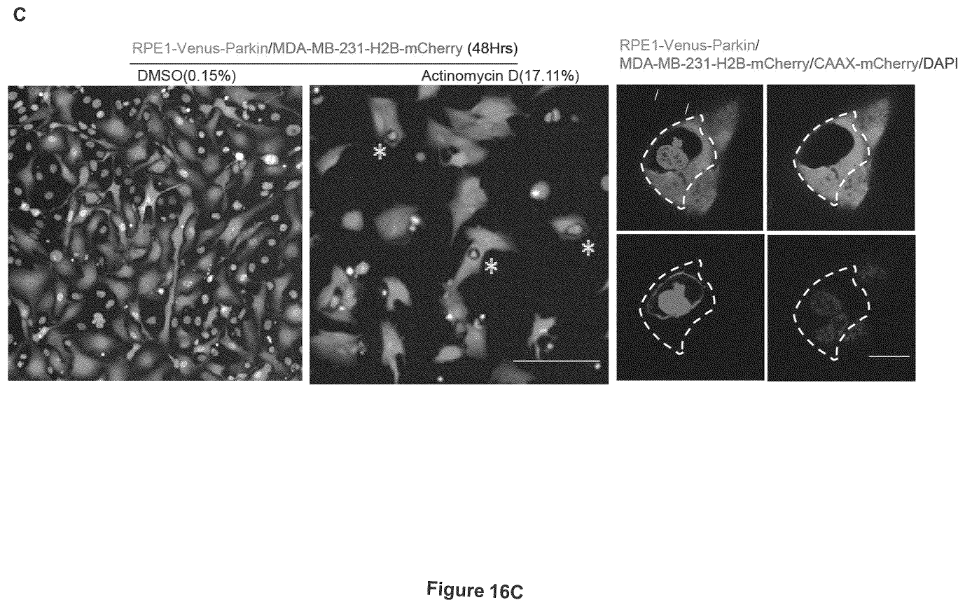

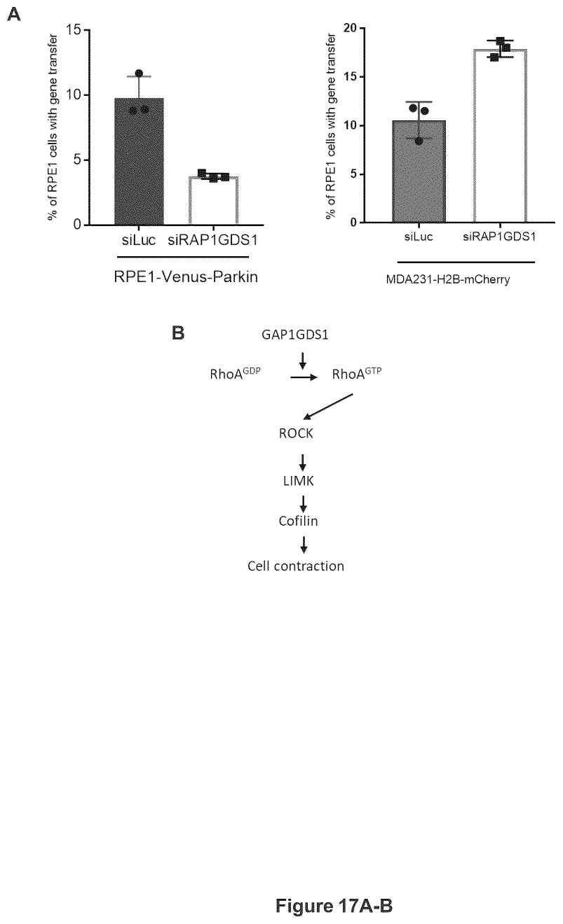

To investigate additional cell signaling pathways critical for tangocytosis, the present inventors generated a high-content screening platform for small-molecule inhibitors of tangocytosis. As noted above, the present inventors have identified Stavudine as a potential tangocytosis inhibitors with IC50 of 1.83 μM. Using this inhibitor as a positive control, the inventors developed a high content imaging-based HTS screening assay using the PerkinElmer Opera Phenix microscope. Briefly, 5×104 donor cells MDA-MB-231-H2BmCherry cells were mixed with same number of RPE-VenusParkin and seeded into the wells of 384-well plates preloaded with DMSO, Stavudine along with a population of various drug candidates. The plate were imaged and analyzed using the custom optimized protocol, including optimized cell segmentation, which can distinguish the overlapping and double positive cells. Our preliminary result suggests that the Z′ factor coefficient of the screening plate is ≥0.69, indicating a fairly robust assay (FIG. 10-17). As noted in Table 2, the present inventors identified a number of small-molecule inhibitors of tangocytosis.

### Example 7: Dual Roles of Actinomycin D, Doxorubicin, Hygromycin B

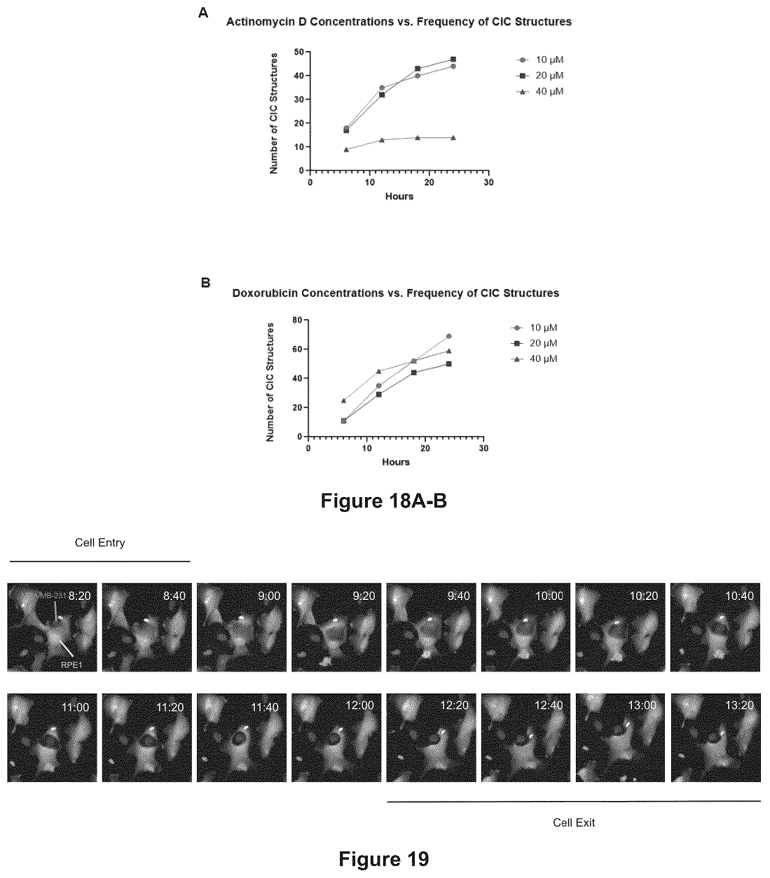

As noted above, Actinomycin D promotes cell entrapment but prevents subsequent gene transfer due to its cytotoxicity against cancer cells, whereas Doxorubicin, another antibiotic for cancer treatment, enhanced cell-in-cell structures while inhibiting gene transfer of the reporter gene. Both drugs had the capability of trapping the tumor cell inside the epithelial cell for extended periods. To optimize these occurrences, MDA-MB-231-H2B-mCherry and RPE1-VEP were co-cultured, and each was treated with 10 μM, 20 μM, and 40 μM concentrations of Actinomycin D and Doxorubicin individually. These cells were studied under live cell imaging for 48 hours which captured images every 20 minutes, allowing visualization for the concentration that induced the most CIC structures. 10 μM Doxorubicin and 20 μM Actinomycin D were the concentrations that produced the highest frequency of CIC structures (FIG. 18).

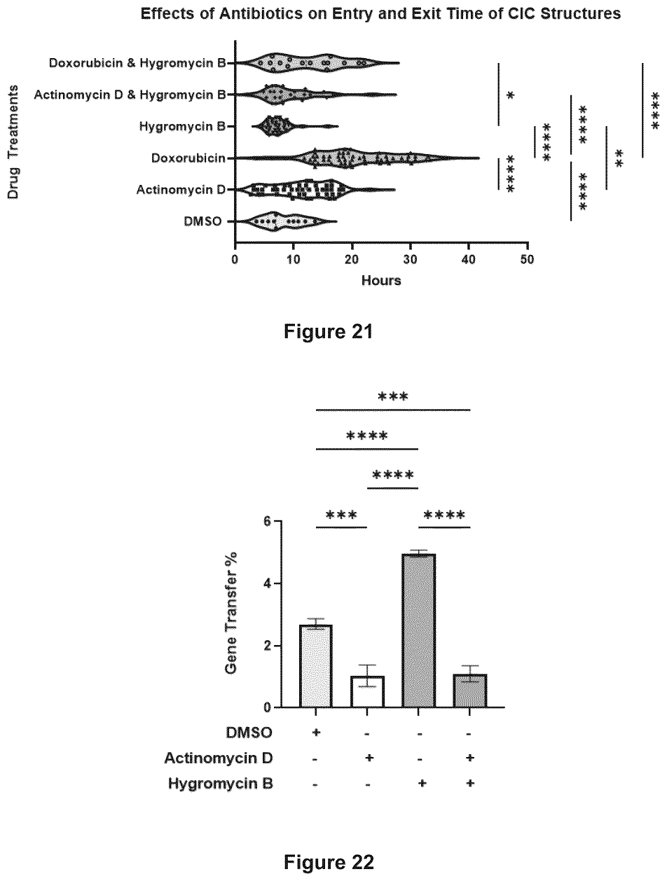

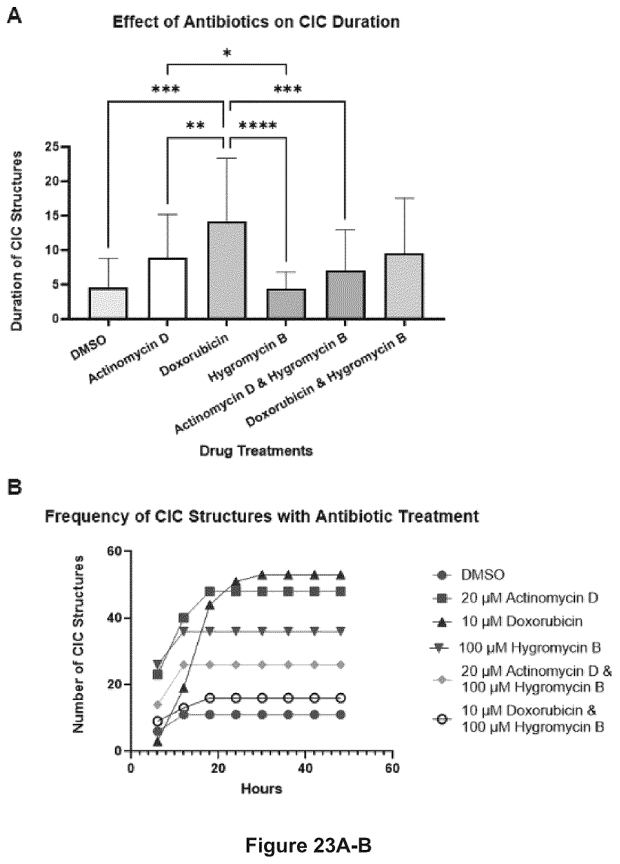

From this information, the duration of these CIC structures in the respective optimal drug concentrations was studied by recording the entry and exit time through 48-hour live cell imaging (FIG. 19). The DMSO control formed CIC structures minimally with an average duration of 4.5 hours and they were concentrated at 5.9-10.4 hours during a 48-hour live cell imaging period (FIG. 21, 23A). 10 μM Doxorubicin had an average duration of 14.24 hours and concentrated at 14-27.7 hours while 20 μM Actinomycin D had an average duration of 8.9 hours and concentrated at 7.8-16.9 hours (FIG. 21, 23A). CIC structures formed with Doxorubicin significantly increased in frequency, duration, and entry/exit time when compared to DMSO-treated co-culture cells (FIG. 21, 23A). Actinomycin D significantly varied in frequency but there was no significance with duration and time occurrence when compared to DMSO. The CIC durations and time occurrences between Actinomycin D and Doxorubicin treatments were statistically significant with a p-value of 0.0006 and >0.0001, respectively (FIG. 21, 23A). 10 μM Doxorubicin treatment resulted in the most CIC structure formation (FIG. 23B).

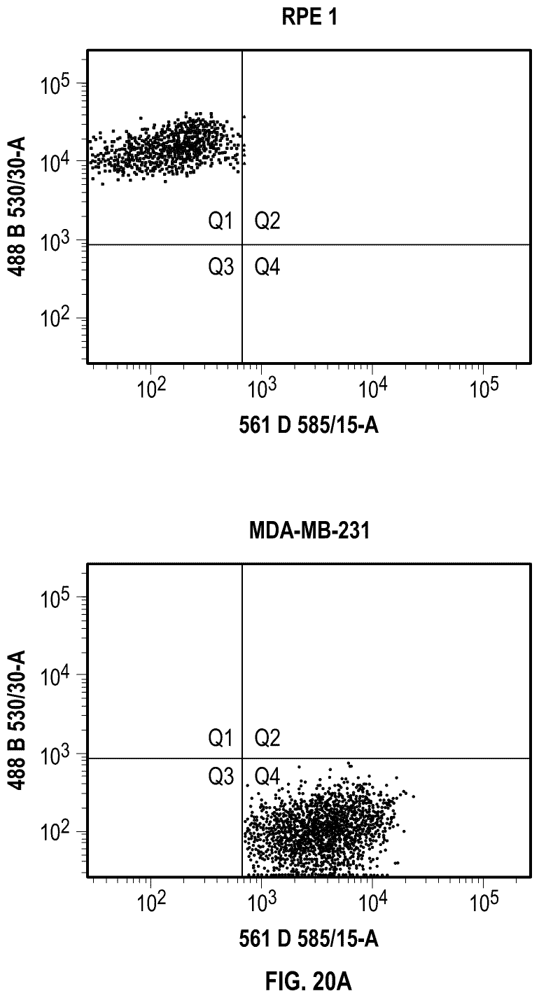

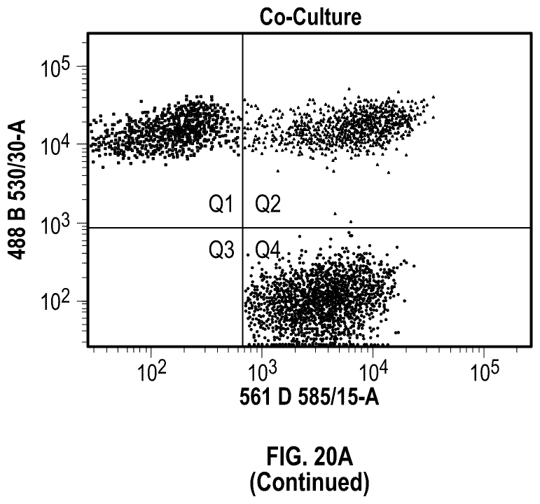

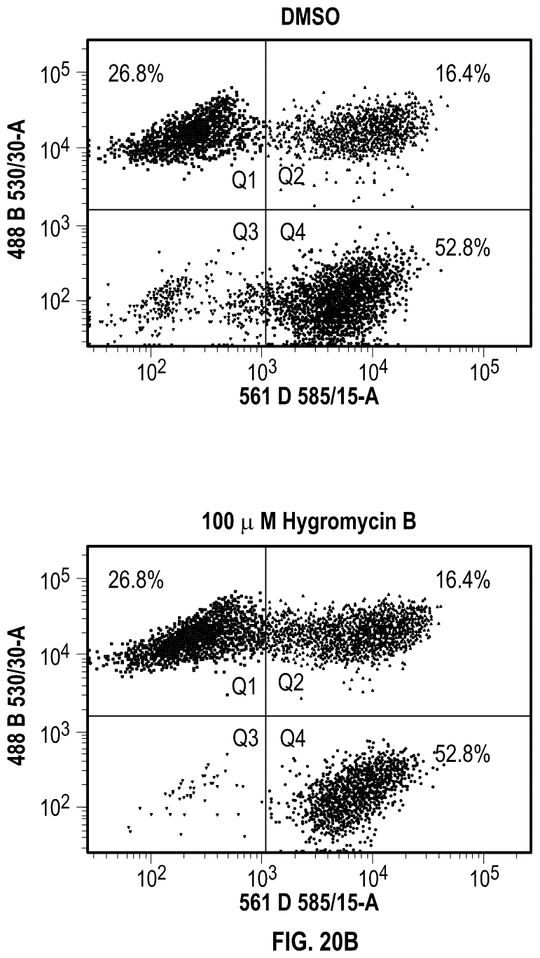

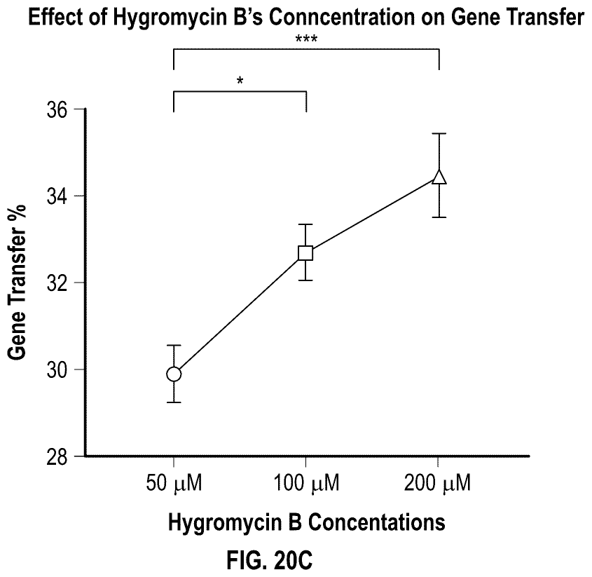

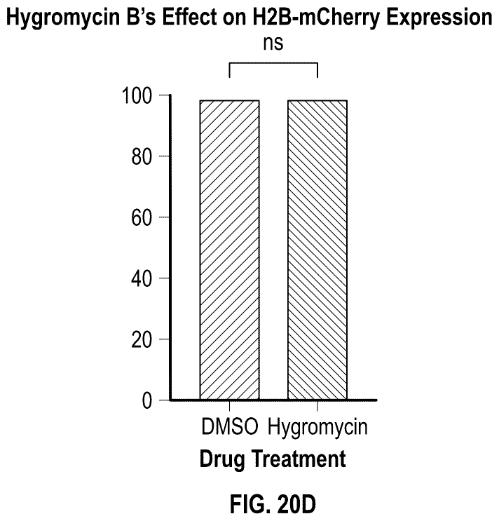

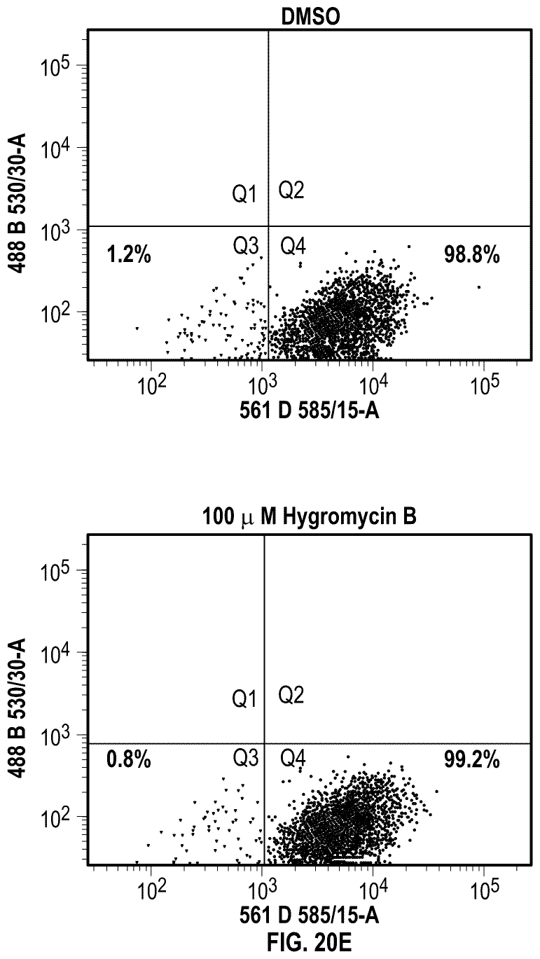

Hygromycin B shows significantly increases gene transfer of H2BmCherry from MDA-MB-231-H2B-mCherry to RPE1-VEP (FIG. 20B). To determine which concentration produced the highest gene transfer percentage, MDA-MB-231-H2B-mCherry and RPE1-VEP were again co-cultured and treated with 50 μM, 100 μM, and 200 μM of Hygromycin B for 48 hours. Through flow cytometry analysis, 100 μM and 200 μM concentration increased gene transfer significantly more than 50 μM with a p-value of 0.0103 (FIG. 20C). There was no statistical significance between 100 μM and 200 μM with a p-value of 0.0695 (FIG. 20C). 100 μM Hygromycin B was chosen for further analysis. To look at this phenomenon in detail, duration, the number of cell-in-cell structures, and entry/exit time were quantified. 100 μM Hygromycin B had an average cell-in-cell structure duration of 4.47 hours and these structures occurred most frequently at 5.1-9.6 hours when looked at in a 48-hour live cell imaging period (FIG. 21). The duration of Hygromycin B was significantly less when compared with Doxorubicin (p-value<0.0001) and Actinomycin D (p-value 0.0211) but had no statistical significance with DMSO-treated co-culture (FIG. 23A). Hygromycin induced CIC structures early in the cell-culture (FIG. 21). Additionally, it was determined that Hygromycin B doesn't enhance gene transfer through amplifying H2B-mCherry gene expression (FIG. 20D, 20E).

Actinomycin D, Doxorubicin, and Hygromycin B had significantly different durations and entry/exit times (FIG. 21, 23A, 23B). To see if a combination of each of these drugs would impact the duration and frequency of the formation of CIC structures, 48 hours of live cell imaging was completed with 100 μM Hygromycin B & 20 μM Actinomycin D and 100 μM Hygromycin B & 10 μM Doxorubicin treatments on co-cultured cells. Hygromycin B and Actinomycin D had an average CIC duration of 7.12 hours and they occurred most frequently at 5.9-13 hours (FIG. 7). Hygromycin B and Doxorubicin had an average CIC duration of 9.54 hours and they occurred most frequently at 2-10 hours and 7.6-17.2 hours (FIG. 21). These treatments decreased the overall number of CIC structures formed when compared to individually treated co-cultures. There was no significant difference in the time occurrence or CIC duration of individually treated Actinomycin D or Hygromycin B vs. treatment together (FIG. 21, 23A). Doxorubicin & Hygromycin B-treated cells seemed to adopt Doxorubicin's duration-lengthening effect. However, no statistical significance was proven when compared to DMSO and Hygromycin B (FIG. 23A). Additionally, the treatment adopted Hygromycin B's early occurrence of CIC structure formation when compared to individually treated Doxorubicin CIC structures (FIG. 21).

Flow Cytometry analysis was used to quantify the gene transfer percentage of DMSO, 20 μM Actinomycin D, 100 μM Hygromycin B, and both treatments together on co-cultured MDA-MB-231-H2B-mCherry and RPE1-VEP. Unfortunately, Doxorubicin-treated co-cultured cells resulted in an assay interference for the red fluorescent protein, and the gene transfer percentage was unable to be measured. Actinomycin D and Actinomycin D with Hygromycin B had similar inhibitory effects of gene transfer with both averaging 1.5% (FIG. 22). Consequently, the gene transfer percentage of these treatments was significantly lower when compared to 100 μM Hygromycin B's treatment alone which resulted in 4.5% gene transfer (FIG. 22).

Latrunculin B is an actin polymerization inhibitor that has been shown to block CIC structure formation as the CIC structure process is mediated by actin cytoskeleton organization with ROCK kinases. Subsequently, Latrunculin B treatment on co-cultured RPE1-VEP and MDA-MB-231-H2BmCherry significantly decreased gene transfer and F actin levels, showing the importance of actin dynamics in CIC structure formations. Hygromycin B has been shown to induce CIC structures with shorter durations while Actinomycin D and Doxorubicin entrap the tumor cell for longer durations. It was hypothesized that Latrunculin B can impact the frequency and duration of CIC structure when treated with the various antibiotics previously studied. Applicant previously described, 3 μM of Latrunculin B treatment resulted in the lowest percentage of gene transfer to RPE cells. As a result, co-cultured RPE1-VEP and MDA-MB-231-H2B-mCherry cells were treated with 3 μM Latrunculin B in combination with 10 μM Doxorubicin, 20 μM Actinomycin D, or 100 μM Hygromycin B and the CIC structures were visualized with the Opera Phenix. No cell-in-cell structure formation occurred in just Latrunculin B treated co-culture or in Latrunculin B in combination with Actinomycin D or Doxorubicin treated cells, reinforcing the importance of actin dynamics when these structures occur (FIG. 21B)

### Example 8: MDASC43's Absence of Gene Transfer but Presence of CIC Structures

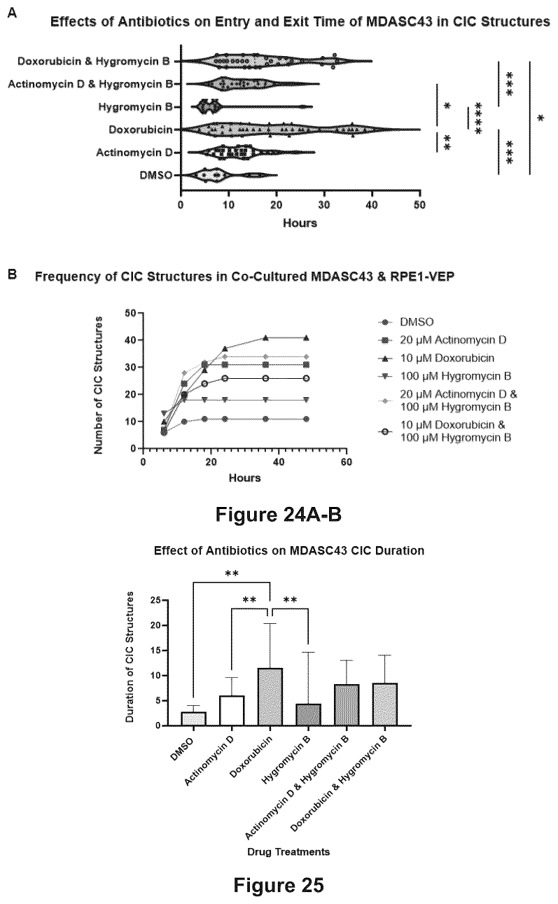

MDASC43 is a single clone of the MDA-MB-231 cell line that shows little to no gene transfer when co-cultured with RPE1-VEP. Previous reports show that CIC structures are an important step in the gene transfer process as it requires direct cell-cell contact. With this logic, MDASC43 should form no CIC structures as no gene transfer is occurring. However, when MDASC43 was co-cultured with RPE1-VEP, the intercellular mosaic structures were observed in DMSO with an average duration of 2.79 hours and concentrated at 6.2-9.1 hours of a 48-hour live cell imaging period (FIG. 24A, 25). In parental RPE1-VEP and MDA-MB-231-H2B-mCherry co-culture, 10 μM Doxorubicin, 20 μM Actinomycin D, and 100 μM Hygromycin B significantly increased the frequency of CIC structures and affected entry and exit times differently. Similar effects were observed in MDASC43. The number of CIC structures was significantly enhanced with all 3 antibiotic treatments. Doxorubicin-treated CIC structures had significantly higher durations while Hygromycin B showed decreased durations when observed with Opera Phenix live cell imaging. 20 μM Actinomycin D-treated MDASC43 & RPE1-VEP formed CIC structures that had an average duration of 6.04 hours and concentrated at 9.2-15.3 hours (FIG. 24A, 25). 10 μM Doxorubicin-treated MDASC43 & RPE1-VEP CIC had an average CIC duration of 11.5 hours, and the structures were concentrated at 13.6-25.1 hours (FIG. 24A, 25). Doxorubicin significantly increased CIC structure duration when compared to DMSO (FIG. 24A, 25). 100 μM Hygromycin B treatment resulted in CIC structures concentrated at 4.7-9.2 hours with an average duration of 4.44 hours (FIG. 24A, 25). Doxorubicin-treated co-culture had a significantly higher CIC duration than Hygromycin B treated co-culture (FIG. 25). 10 μM Doxorubicin treatment resulted in the most CIC structure formation (FIG. 24B).

Furthermore, the CIC structure time occurrences and durations were measured when treated with a combination of antibiotics. 10 μM Doxorubicin and 100 μM Hygromycin B treated co-culture in combination resulted in CIC structures concentrated at 12.5-21.2 hours with an average duration of 8.6 hours (FIG. 24A, 25). 20 μM Actinomycin D and 100 μM Hygromycin B treated co-culture in combination resulted in CIC structures concentrated at 8.9-17.2 hours with an average duration of 8.33 hours (FIG. 24A, 25 Similar to the parental cell lines, there was no significant difference in the time occurrence or CIC duration of individually treated Actinomycin D or Hygromycin B vs. treatment together (FIG. 24A, 25). Additionally, Doxorubicin and Hygromycin B treated co-culture cells in combination seem to adopt Doxorubicin's duration lengthening effect as seen in the parental cell lines but again, no statistical significance was proven when compared to DMSO and Hygromycin B treated CIC structures (FIG. 24A).

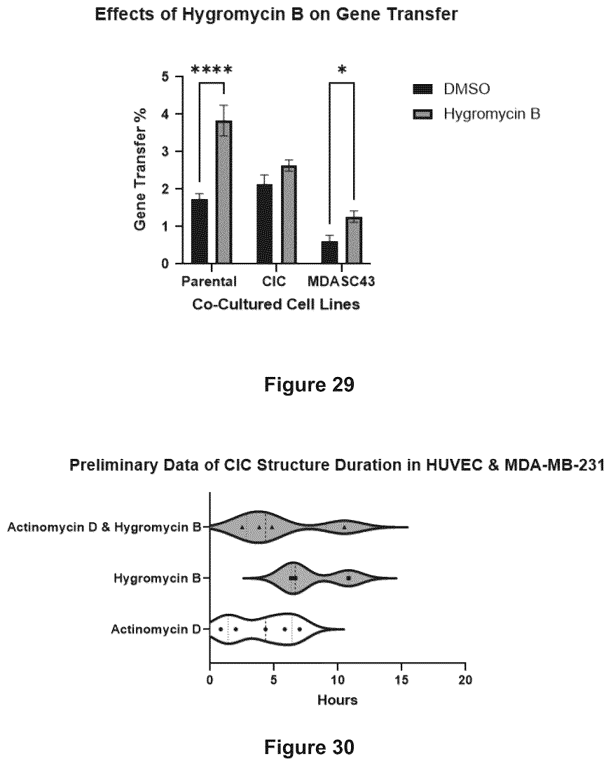

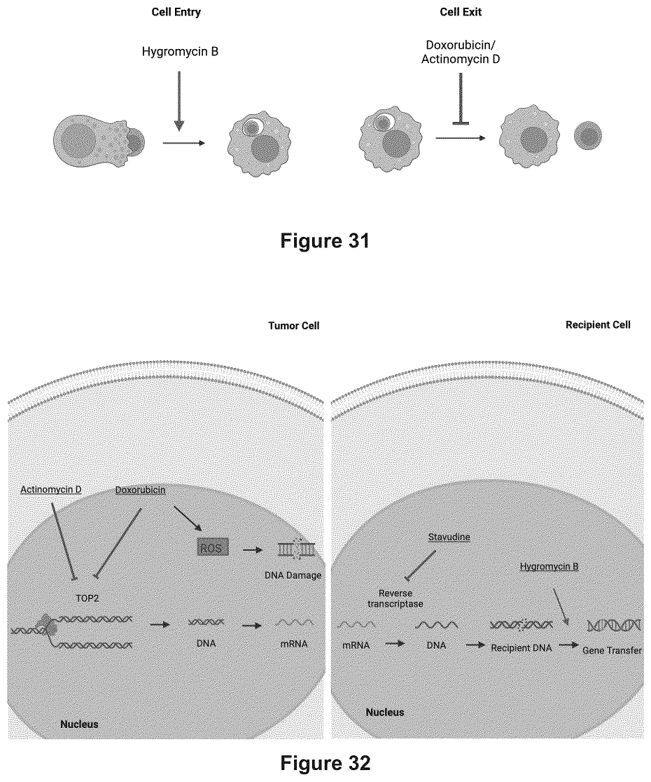

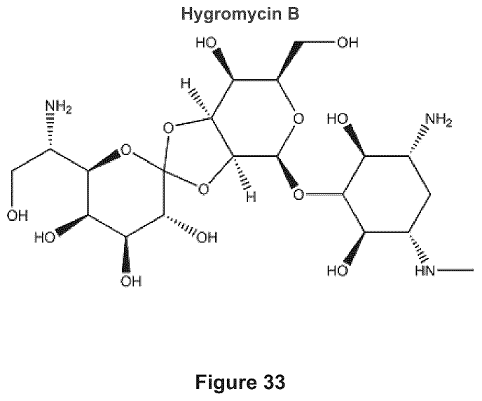

Hygromycin B has been observed to increase gene transfer in parental RPE1-VEP and MDA-MB-231-H2B-mCherry co-culture. To study if the antibiotic would have the same effect on co-cultured MDASC43 and RPE1-VEP, this was treated with 100 μM Hygromycin B for 48 hours. Hygromycin B significantly increased the gene transfer percentage of H2B-mCherry to RPE1-VEP from MDASC43 to RPE1-VEP (FIG. 29).

### Example 9: Cell Fates after Tangocytosis

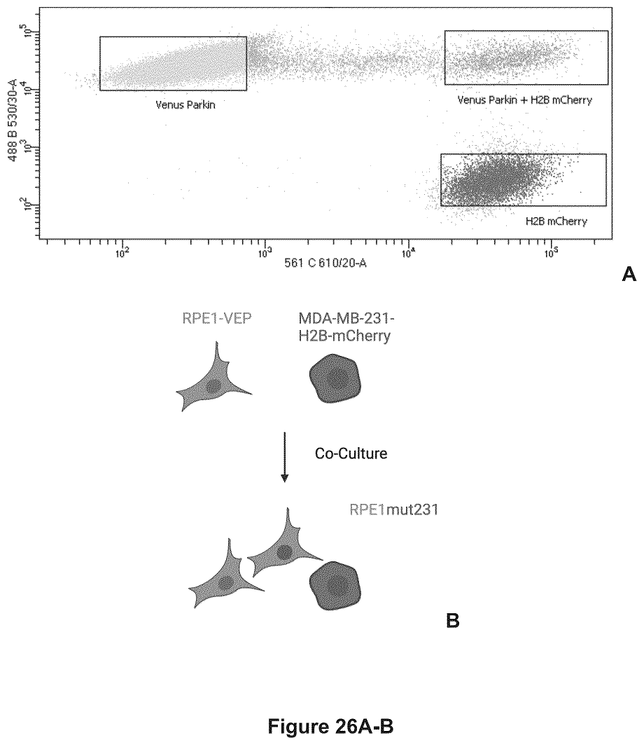

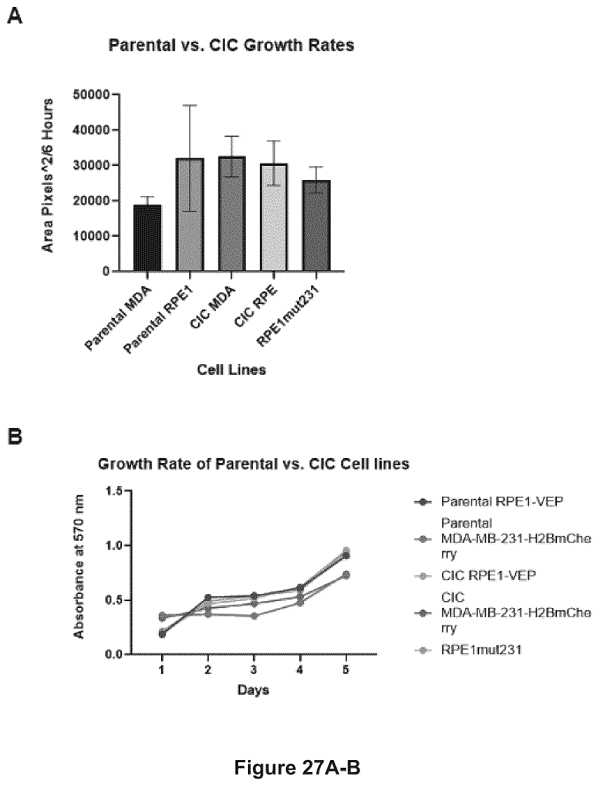

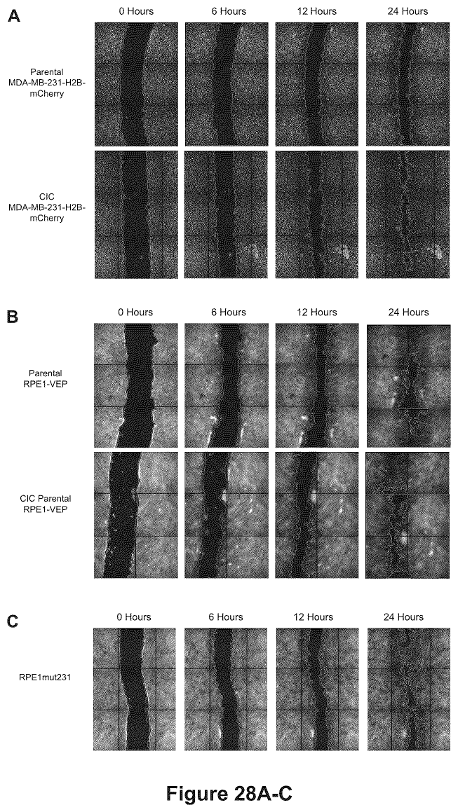

Similarly to entosis, after the MDA-MB-231 cell dives into RPE1, the cell fates that have been observed are either the survival or death of both or one of the cells. However, the growth of the MDA-MB-231 cell, “CIC MDA”, RPE1 “CIC RPE”, and RPEmut231 after a CIC structure formation has been scarcely studied. After co-culturing MDA-MB-231-H2B-mCherry and RPE1-VEP for 48 hours, the double-positive cells, RPE1mut231, were sorted out with BD FACSAria Fusion (FIG. 26). The double-positive cells were incubated for 1 week to reach confluency until a second round of cell sorting was done to separate the CIC MDA, CIC RPE, and RPE1mut231. To perform a wound healing experiment, 1E5 cells/ml of parental RPE1-VEP, parental MDA-MB-231-H2B-mCherry, CIC RPE, CIC MDA, and RPE1mut231 were seeded into a 96-well plate and reached full confluency. A scratch was made in each well of the cell lines and the growth was visualized with 48-hour live cell imaging (FIG. 28A-C). After analyzing the live cell imaging at 6-hour time points with the “Wound Healing Size Tool” ImageJ plugin, the area pixels\hat{ } 2/6 hours was calculated, and no significant difference was observed. This was further confirmed with crystal violet staining performed for 5 days which showed no significant differences between the parental and CIC cell lines in terms of growth.

Additionally, co-cultured CIC RPE1 and CIC MDA were treated with 100 μM Hygromycin B to see if the gene transfer enhancement effect would be observed. Gene transfer was analyzed with Flow Cytometry. However, there was no statistical significance between DMSO and Hygromycin B in terms of gene transfer percentage in the CIC cell lines (FIG. 29).

### Example 10: Tangocytosis in Human Umbilical Vein Endothelial Cells

Human umbilical vein endothelial cells (HUVEC) are isolated from the inner surface of blood vessels which are a target for breast cancer cells during the metastatic process. It would be significant to in vivo studies if the above results could be observed in this cell line. To study this further, preliminary data was obtained by co-culturing HUVEC with MDA-MB-231-H2B-mCherry cells and treating the cells with 100 μM Hygromycin B, 20 μM Actinomycin D, and a combination of 100 μM Hygromycin B and 20 μM Actinomycin D. By studying 1 focus field due to a limited number of cells, the number of CIC structures observed were restricted but still valuable. The duration and time occurrences were observed. In each treatment, there was no significant difference in the frequency of CIC structures. In 100 μM Hygromycin B treatment, the average CIC structure duration was 3 hours with the structures concentrated at 6.4-9.4 hours (FIG. 18). In 20 μM Actinomycin D treatment, the average CIC structure duration was 2.1 hours with the structures concentrated at 2.9-5.1 hours (FIG. 18). In 100 μM Hygromycin_B and 20 μM Actinomycin D treatment, the average CIC structure duration was 4.2_hours and the structures were concentrated at 3.3-7.5 hours (FIG. 18). There was no statistical significance between durations and entry/exit times of each antibiotic_treatments.

### Example 11: The Effects of Antibiotics on Cell Entry and Exit

Actinomycin D and Doxorubicin as described herein are able to induce CIC structures. Hygromycin B has been shown to significantly increase CIC structures and enhance gene transfer of the reporter gene, H2B-mCherry, from MDA-MB-231 to RPE1-VEP when compared to DMSO control. Hygromycin B's effect of gene transfer enhancement is also seen in co-cultured MDASC43 and RPE1-VEP, emphasizing its significant effect on the tangocytosis process. From the studies examining the entry and exit times of CIC structures in each antibiotic treatment of parental cell line co-cultures, CIC structures in 10 μM Doxorubicin had significantly longer durations when compared to 100 μM Hygromycin B treated cells and DMSO. These results were also observed in MDASC43 and RPE1-VEP co-culture. CIC structures in 20 μM Actinomycin D had significantly longer durations than 100 μM Hygromycin B but not DMSO in parental cell line co-culture. This suggests Doxorubicin and Actinomycin D allow the RPE1 cell to lock in the MDA-MB-231 cell in a CIC structure for longer periods without gene transfer, inhibiting cell exit. In addition, Hygromycin B induces short cell entrapments early in the co-culture that promote gene transfer, suggesting the promotion of cell collisions and therefore, cell entry and actin rearrangement. Hygromycin B's mechanism of the enhancement of gene transfer has been shown to not include a pathway that amplifies H2B-mCherry gene expression.

When co-cultured MDA-MB-231-H2B-mCherry and RPE1-VEP were treated with both 20 μM Actinomycin D and 100 μM Hygromycin B, there was no statistical significance in terms of CIC duration and time occurrences when compared to individually treated co-cultures. These results were also observed in MDASC43 and RPE1-VEP co-cultured cells. However, when the gene transfer percentage was calculated through flow cytometry, the gene transfer percentage calculated from co-cultures treated with both Actinomycin D and Hygromycin B was similar to the gene transfer percentage of just Actinomycin D treated co-cultures of parental cell lines. This suggests that Actinomycin D's effect of gene transfer inhibition has a dominant effect over Hygromycin B's gene transfer enhancement effects. In addition, when co-cultures were treated with both 10 μM Doxorubicin and 100 μM Hygromycin B, the CIC structures had longer durations that were similar to Doxorubicin's effect. These results were again, seen in MDASC43 and RPE1-VEP co-cultured cells but statistical significance wasn't proven when compared to DMSO-treated or Hygromycin B-treated CIC structures. This can suggest Doxorubicin's potent role as an inhibitor of cell exit. Latrunculin B showed virtually no CIC structures, highlighting the importance of actin rearrangement in CIC structures.

### Example 11: Independence of Gene Transfer & CIC Structures

Tangocytosis is postulated to be an important step in metastasis where it includes cell-in-cell structure formation and gene transfer. Actinomycin D and Doxorubicin inhibit gene transfer but both antibiotics significantly increase the frequency of CIC structures. This suggests that Doxorubicin and Actinomycin D induce a pathway where H2B-mCherry gene transfer is inhibited. A possible explanation may be that the mRNA from the reporter gene is being integrated into an incompetent area of the recipient gene locus. Additionally, RPE1-VEP was shown to entrap MDASC43 cells, although this cell line inhibits gene transfer. Treatments of 20 μM Actinomycin D and 10 μM Doxorubicin significantly increased the frequency of CIC structures in this co-culture while continuing to inhibit the gene transfer of H2B-mCherry. This suggests that gene transfer may not always be directly correlated with CIC structure formation and these occurrences may be separate processes.

Hygromycin B induces both gene transfer and CIC structures in parental cell line co-culture and MDASC43 co-cultured with RPE1-VEP. MDASC43 is normally known to inhibit gene transfer but when treated with 100 μM Hygromycin B, the cell line forms frequent CIC structures with RPE1-VEP while showing increased gene transfer, suggesting a positive correlation between the CIC structure formation and gene transfer. A possible explanation may be that Hygromycin B leads to an increase in gene transfer as it induces a pathway that increases the competency of the recipient cell to allow for the uptake of the donor mRNA. In addition, due to its absence of cytotoxicity and both, RPE1 and MDA-MB-231, having a Hygromycin B resistant gene, Hygromycin B can potentially have both an increase in CIC structures and gene transfer.

### Example 12: Proposed Mechanism of Gene Transfer

As discussed, it has been postulated that intercellular gene transfer occurs through an mRNA intermediate, direct cell-cell contact, and reverse transcription of the donor cell mRNA to DNA to subsequently integrate the DNA into the host genome. In the tumor cell, DNA replication and transcription occur to result in the mRNA intermediate. Actinomycin D and Doxorubicin likely inhibit topoisomerase 2. Topoisomerase 2 is known to play an important role in DNA replication to manage supercoils by inducing double-stranded breaks. Actinomycin D and Doxorubicin likely inhibit the re-ligation of DNA strands after a double-stranded break, leading to DNA damage with no repair. Additionally, Doxorubicin induces reactive oxygen species that further promote DNA damage.

The tumor cell mRNA enters the recipient cell where it is transcribed to DNA through reverse transcription. Stavudine likely inhibits the reverse transcriptase, blocking a vital step and leading to decreased gene transfer. After DNA is transcribed, it is ready to be integrated into the host genome. Hygromycin B likely mitigates tumor cell DNA integration by increasing the competency of the recipient cell, leading to enhanced gene transfer.

### Example 13: Cell Fates and In Vivo Application

After studying the growth rate of CIC MDA-MB-231, CIC RPE1-VEP, and RPE1mut231 and comparing them to parental cell lines, no significant findings were produced. This suggests CIC structures don't play an important role in increasing the proliferation of the recipient or donor cell after cell entrapment. Vascular endothelial cell lines directly interact with the bloodstream and play an important role in processes such as angiogenesis, vascular permeability, and leukocyte trafficking. As a result, preliminary studies showed that CIC structures can be induced by Actinomycin D, Hygromycin B, and a treatment of both Actinomycin D and Hygromycin B. Even though significant results weren't seen in the preliminary data, this expands the breadth of the use of these antibiotics in in-vivo circumstances to model tumor metastasis as it migrates to the vascular system.

### Example 14: Materials and Methods

Constructs, Stable cell line, and Cell culture: The Parkin gene was inserted into retroviral expression vector pREX-Venus-DEST-IRES Blasticidin and mCherry-tagged H2B was inserted into pREX-IRES-Hygromycin as described previously. Cytosolic TagBFP was expressed using pCRISPRi/a-V2 (Addgene #84832). Two strategies were employed for gene knockdown: for mCherry mRNA knockdown, hairpin

(5′-CCGGGTGGGAGCGCGTGATGAACTTCTCGAGAAGTTCATCACGCG
CTCCCACTTTTTG-3′)

was cloned into pLKO.1-TRC (Addgene #10878); for ROCK1/2 mRNA knockdown, the validated commercial siRNAs against ROCK1 (5′-GGUUAGGGCGAAAUGGUGUtt-3′) (SEQ ID NO. 1) or ROCK2 (5′-GGAGAUUACCUUACGGAAAtt-3′) (SEQ ID NO. 2) were purchased from ThermoFisher. The stable cell lines were constructed as described previously. All cell lines were cultured in Dulbecco's modified Eagle medium (DMEM) supplemented with 10% FBS, 2 mM glutamine, 100 U/mL penicillin, and 100 mg/mL streptomycin at 37° C. with 5% CO2 incubation.

Cell synchronization: Cells were synchronized at G1/S by single thymidine treatment for 24 hours. Cells arrested at G1/S were released into medium containing RO3306 for 19 hours and collected by shaking off to obtain mitotic cells.

Flow cytometric analysis: 1×10⁵ RPE1-Venus-Parkin cells alone or along with MDA-MB-231-H2B-mCherry cells were seeded on 12-well plate and incubated for 2 days. These cycling populations were digested with trypsin/EDTA and then subjected to flow cytometric analysis after being resuspended with 0.5 mL DMEM.

Karyotype analysis: Cytogenetic analysis was performed on ten G-banded metaphase spreads of RPE1 and RPE1mut231 cell lines at passage 20. Karyotype analysis was performed by Karyologic, Inc (Research Triangle Park, NC).

Retroviral integration site analysis: Genomic DNA from MDA-MB-231 or RPE1 cells was isolated from subconfluent culture cells 50 growing on 6 cm plate using a QIAamp DNA blood kit (QIAGEN). A Universal GenomeWalker™ 2.0 kit (TakaRa, USA) was used to design and amplify the MoLV LTR junctional fragments from both cell lines prior to subcloning into a TA-cloning vector pCR2.1 (ThermoFisher) and sequencing.

Live cell imaging: For long-term imaging using an ImageXpress microscope, donor cells and recipient cells were seeded at a 1:1 ratio on glass-bottomed dishes (Mattek, Ashland, MA) in a 150 μL complete FluoroBrite DMEM and incubated/treated as shown in figure legends. All live microscopy was performed in an incubation chamber at 37° C., with 5% CO2 and for long-term imaging media was overlaid with mineral oil. Fluorescent images were acquired every 0.5 or 1 hour, and data analysis was performed with Image J software. Confocal images were acquired on a Nikon AIR Confocal and TIRF using a 100× (NA 1.45) objective or Opera Phenix (PerkinElmer) using a 40× water objective. For immunofluorescence microscopy, cells were fixed with 4% paraformaldehyde. Immunofluorescence microscopy was performed as described previously.

Quantification and Statistical Analysis: The donor to recipient cell ratio (Rd/r) was calculated as the number of donor cells (Q3+Q4) divided by the number of recipient cells (Q1+Q2). The trendline and equation were generated using a built-in statistical tool in Microsoft Excel. To determine the effect of Rd/r ranges on gene transfer, the training data was generated as described in Methods and Materials and followed by one-way ANOVA and Post Hoc analysis. Unpaired t-tests were used to calculate p-values for each set of compared results. To determine the effect of donor to recipient cell ratio (Rd/r) on gene transfer frequency, we used built-in statistical tool in Microsoft Excel to generate the trendline and equation. The training data were generated using the original data, original data with 90% SD, and original data with 110% SD to determine the effect of Rd/r ranges on gene transfer. These data were analyzed in GraphPad Prism using one-way ANOVA plus Post Hoc analysis. All other calculations, including average, SD, and P values, were performed using GraphPad Prism software (GraphPad Software, Inc.).

Cell Culture II: MDA-MB-231-H2B-mCherry and RPE1-VenusParkin were each cultured in a 10 cm dish in Dulbecco's Modified Eagle Medium (DMEM) supplemented with 10% fetal bovine serum (FBS), penicillin, streptomycin (100 IU/ml and 100 mg/ml, respectively), and L-Glutamine in a humidified incubator at 37 degrees Celsius with 5% CO2 atmosphere.

Cell Sorting II: 1E5 cells/ml of Parental MDA-MB-231-H2B-mCherry and Parental RPE1-VenusParkin were seeded 1:1 ratio in a 10 cm dish and co-cultured in an incubator for 48 hours. Double positive cells expressing both H2B-mCherry and VenusParkin (RPE1mut231) were separated into a well of a 96-well plate using a cell sorter (BD FACSAria Fusion Cell Sorter, BD Biosciences). Once confluent, Trypsin-EDTA (0.25%) (ThermoFisher) was used to detach cells from the plate and subsequently transferred to a 48-well plate, 24-well plate, 6-well plate, and 10 cm dish. From this culture of cells, a second round of cell sorting was completed to separate CIC MDA-MB-231-H2BmCherry, CIC RPE1-VEP, and RPE1mut231.

MDASC43: A single clone of the MDA-MB-231 cell line (SC43) was made by limiting dilution. MDA-MB-231 cells were digested with Trypsin/EDTA and cells were resuspended in complete DMEM medium. The cells were counted to have 8 cells/ml and cells were subsequently transferred to a 96-well plate by adding 100 μL of cell suspension to each well. After 10 days, the wells with cells were identified by Opera Phenix. The cells were amplified by splitting the cells into a larger culture plate. Co-culture assay was performed by growing an equal number of MDA231 single clone cells and RPE1 cells for 48 hours. Flow cytometry was performed to determine the gene transfer ratio.

Gene Transfer & Expression Quantification II: 1E5 cells/ml of each cell line were counted: Parental MDA-MB-231-H2B-mCherry, Parental RPE1-VenusParkin, CIC MDA-MB-231-H2BmCherry, CIC RPE1-VEP, and MDASC43. In a 12-well plate, the following combinations of cell lines were co-cultured with treatments including: Dimethyl sulfoxide (DMSO) for control, 100 μM Hygromycin, 20 μM Actinomycin D, 10 μM Doxorubicin, 10 μM Doxorubicin & 100 μM Hygromycin B, and 20 μM Actinomycin D & 100 μM Hygromycin B. In each well, the following amounts were added: 2 mL of 1E5 Parental MDA-MB-231-H2B-mCherry & Parental RPE1-VenusParkin were co-cultured, 2 mL of 1E5 CIC MDA-MB-231-H2BmCherry & CIC RPE1-VEP, 2 mL 1E5 MDASC43 & RPE1-VEP. Each treatment had 3 repeats. The 12-well plate was placed in the incubator for 48 hours with DMEM suspension. The cells were suspended in DMEM and the gene transfer percentage was quantified using flow cytometry (BD Accuri C6, BD Biosciences). This procedure was repeated to study H2B-mCherry expression by culturing MDA-MB-231-H2BmCherry treated with DMSO and 100 μM Hygromycin B.

Live Cell Imaging II: 1E5 cells/ml of MDA-MB-231-H2B-mCherry and RPE1-VenusParkin were seeded 1:1 while suspended in DMEM in respective wells of a 96-well sterile ViewPlate microplate (clear bottom). Each well was treated with a different drug treatment or a combination from the following list: DMSO (control), 10 μM Doxorubicin, 20 μM Actinomycin D, 100 μM Hygromycin B, 3 μM Latrunculin B. 48-hour live cell imaging was performed by using the Opera Phenix (Perkin Elmer). A 20× air objective was used and the cells were kept at 37° C., with 5% CO2. Pictures were taken every 20 minutes. This process was repeated with cocultures of MDASC43 & RPE1-VEP and MDA-MB-231-H2B-mCherry & HUVEC treated with various drugs. The duration of CIC structures was calculated by observing the entry and exit times.

Wound Healing Assay II: In each well of the 96-well sterile ViewPlate microplate (clear bottom), 1E5 cells/ml of each cell line were seeded, and suspended in DMEM. Each respective cell line had 3 repeats. The plate was placed in an incubator for 7 days until all cell lines reached full confluency. A 200 μL pipette tip was connected to a 200 μL pipette to create a scratch in the middle of each well. After the scratch, 48-hour live cell imaging was performed by using the Opera Phenix (Perkin Elmer). To calculate the growth of each cell line, the “Wound Healing Size Tool” ImageJ plugin was used to measure the area at time points of 6, 12, and 24 hours.

Crystal Violet Staining II: In 24 well plates, 1E4 cells/ml of each cell line were seeded with DMEM suspension. Each respective cell line had 4 repeats. After each day, the corresponding wells were washed with Dulbecco's Phosphate-Buffered Saline (DPBS). The wells were treated with 2 mL of 70% ethanol and incubated at room temperature for 10 minutes. After 10 minutes, each well was washed with DPBS and maintained in DPBS until 6 days were reached and all cells were fixed with ethanol. 1 mL of Crystal Violet was pipetted into each well and incubated at room temperature for 10 minutes. All plates were washed with tap water twice by immersion in a large beaker. The plates were drained upside down and ensured no residual water. 1 mL of 1% Sodium dodecyl sulfate (SDS) was pipetted into each well to solubilize the stain. SpectraMax iD5 Multi-Mode Microplate Reader (Molecular Devices) was used to measure the absorbance of each well at 570 nm.

Statistical Analysis: Statistical analysis of DMSO vs. Hygromycin B on various co-cultures was done using two-way ANOVA tests to determine significance through p values. Statistical analysis between different antibiotic treatments for frequency, duration, and gene transfer was calculated with one-way ANOVA tests. One-way ANOVA tests were utilized to determine the significance of entry and exit times between different antibiotic treatments in both parental co-cultures and MDASC43 and RPE1-VEP co-cultures. To determine the significance of H2B-mCherry Gene Expression between parental vs. CIC, unpaired t-tests were calculated.

TABLE 1
Effect of donor to recipient cell ratio (Rd/r) on gene transfer frequency.

| Rd/r ranges | 0.5 | 0.6 | 0.7 | 0.8 |
|---|---|---|---|---|
| lgRd/r | −0.301029996 | −0.22184875 | −0.15490196 | −0.096910013 |
| original frequency of | 34.66744751 | 35.76531442 | 36.54511321 | 37.11067057 |
| transferred cells(P1) | | | | |
| P1*0.98 | 35.36079646 | 36.48062071 | 37.27601547 | 37.85288398 |
| P1*1.02 | 33.97409856 | 35.05000813 | 35.81421094 | 36.36845716 |
| Rd/r ranges | 0.9 | 1 | 1.1 | 1.2 |
| lgRd/r | −0.045757491 | 0 | 0.041392685 | 0.079181246 |
| original frequency of | 37.52481643 | 37.828 | 38.04752813 | 38.20254178 |
| transferred cells(P1) | | | | |
| P1*0.98 | 38.27531276 | 38.58456 | 38.80847869 | 38.96659262 |
| P1*1.02 | 36.7743201 | 37.07144 | 37.28657756 | 37.43849094 |
| Rd/r ranges | 1.3 | 1.4 | 1.5 | 1.6 |
| lgRd/r | 0.113943352 | 0.146128036 | 0.176091259 | 0.204119983 |
| original frequency of | 38.306874 | 38.37077788 | 38.40201653 | 38.40657554 |
| transferred cells(P1) | | | | |
| P1*0.98 | 39.07301148 | 39.13819343 | 39.17005686 | 39.17470705 |
| P1*1.02 | 37.54073652 | 37.60336232 | 37.6339762 | 37.63844403 |
| Rd/r ranges | 1.7 | 1.8 | 1.9 | 2 |
| lgRd/r | 0.230448921 | 0.255272505 | 0.278753601 | 0.301029996 |
| original frequency of | 38.38914278 | 38.35344023 | 38.30245859 | 38.23862655 |
| transferred cells(P1) | | | | |
| P1*0.98 | 39.15692564 | 39.12050904 | 39.06850776 | 39.00339908 |
| P1*1.02 | 37.62135993 | 37.58637143 | 37.53640942 | 37.47385402 |
| Rd/r ranges | 2.1 | 2.2 | 2.3 | 2.4 |
| lgRd/r | 0.322219295 | 0.342422681 | 0.361727836 | 0.380211242 |
| original frequency of | 38.16393509 | 38.08003017 | 37.98828289 | 37.88984319 |
| transferred cells(P1) | | | | |
| P1*0.98 | 38.92721379 | 38.84163077 | 38.74804854 | 38.64764006 |
| P1*1.02 | 37.40065639 | 37.31842957 | 37.22851723 | 37.13204633 |

TABLE 2
Small Molecule inhibitors.

| Inhibitor | MOA |
|---|---|
| Clofarabine | DNA Synthesis |
| Clorprenaline HCL | Adrenergic Receptor |
| Nedaplatin | DNA Synthesis |
| Nitroxoline | Antibiotic |
| Chloroxine | Antibiotic |
| Actinomycin D | Apoptosis Inducers |
| Mitomycin C | Apoptosis Inducers |
| Ciclopirox ethanolamine | ATPase |
| Ciclopirox | ATPase, Anti-infection |
| VX-680 (MK-0457, Tozasertib) | Aurora Kinase |
| Ponatinib (AP24534) | Bcr-Abl |
| Nisoldipine | Calcium Channel |
| Atracurium Besylate | Cholinergic receptor |
| Roscovitine (Seliciclib, CYC202) | Cyclin-Dependent Kinases |
| LEE011 | Cyclin-Dependent Kinases |
| Mycophenolate Mofetil | Dehydrogenase |
| Mycophenolic acid | Dehydrogenase |
| Vidofludimus | Dehydrogenase |
| Methotrexate | DHFR |
| Pralatrexate | DHFR |
| Pyrimethamine | DHFR |
| Pemetrexed | DHFR |
| Teriflunomide | DHODH |
| Thio-TEPA | DNA Alkylating |
| Cytarabine hydrochloride | DNA Synthesis |
| Raltitrexed | DNA Synthesis |
| VRT752271 | ERK |
| Amoxapine | GlyT |
| Raltegravir (MK-0518) | HIV Integrase |
| S/GSK1349572 | HIV Integrase |
| GSK1349572 sodiuM salt | HIV Integrase |
| Amprenavir (agenerase) | HIV Protease |
| Atazanavir | HIV Protease |
| Darunavir | HIV Protease |
| Darunavir Ethanolate | HIV Protease |
| Zidovudine | HIV Reverse Transcriptase |
| Stavudine (d4T) | HIV Reverse Transcriptase |
| GS-7340(Tenofovir) | HIV Reverse Transcriptase |
| Tenofovir Disoproxil Fumarate | HIV Reverse Transcriptase |
| Zalcitabine | HIV Reverse Transcriptase |
| Ganetespib (STA-9090) | HSP |
| Diacerein | IL Receptor |
| Trametinib (GSK1120212) | MEK1/2 |
| Trametinib DMSO solvate | MEK1/2 |
| Pimasertib (AS-703026) | MEK1/2 |
| AZD6244 (Selumetinib) | MEK1/2 |
| Ridaforolimus | mTOR |
| (Deforolimus, MK-8669) | |
| Zotarolimus(ABT-578) | mTOR |
| Domiphen Bromide | Others |
| BMN 673 | PARP |
| Anagrelide HCl | PDE |
| BI6727 (Volasertib) | PLK |
| Hexachlorophene | Potassium Channel |
| Pemetrexed disodium | Thymidylate Synthase |
| hemipenta hydrate | |
| Irinotecan hydrochloride | Topoisomerase |
| Etoposide | Topoisomerase |
| Irinotecan | Topoisomerase |
| Irinotecan HCl Trihydrate | Topoisomerase |
| Epirubicin HCl | Topoisomerase |
| Doxorubicin | Topoisomerase |
| Doxorubicin (Adriamycin) HCl | Topoisomerase |
| Sunitinib | VEGFR |
| Sunitinib malate | VEGFR |
| Dovitinib Dilactic acid | VEGFR |

1-4. (canceled)

5. A method of inhibiting horizontal gene transfer (HGT), comprising the step of:
contacting a donor cell and/or a recipient cell with at least one compound selected from: Actinomycin D, Doxorubicin, or a combination of the same.

6. The method of claim 5, wherein said donor cell and/or a recipient cell is a human donor cell and/or a recipient cell.

7. A method of treating cancer in a human subject in need thereof, comprising the step of administering a therapeutically acceptable amount of at least one compound selected from:
Doxorubicin, or a combination of the same;
wherein said compound inhibits horizontal gene transfer (HGT) in the subject.

8-45. (canceled)

46. A method of treating cancer comprising the steps of:
establishing a subject having a donor cancer cell, in contact with a recipient cell wherein said donor cancer cell and said recipient cell are capable of forming an intercellular mosaic structure resulting in the entrapment of said donor cancer cell by said recipient cell; and
administering a therapeutically effective amount of a target inhibitor to said subject in need thereof that inhibits formation of said intercellular mosaic structure or horizontal genetic transfer (HGT) between said donor cancer cell and said recipient cell.

47. The method of claim 46, wherein said step of treating comprises inhibiting metastasis of said donor cancer cell.

48. The method of claim 46, wherein said recipient cell is an epithelial recipient cell.

49. The method of claim 46, wherein said subject comprises a mammal.

50. The method of claim 49, wherein said mammal comprises a human.

51. The method of claim 46, wherein said target inhibitor is selected from the group consisting of: a ROCK1/2 inhibitor, a ROCK1 inhibitor, an actin polymerization inhibitor, a CDC42 inhibitor, a RAP1GDS1/SmgGDS inhibitor or a combination of the same.

52. The method of claim 51, wherein said ROCK1/2 inhibitor comprises ROCK kinase inhibitor Y27632.

53. The method of claim 52, wherein said ROCK1/2 inhibitor comprises an siRNA configured to inhibit expression of ROCK1/2 in said epithelial recipient cell.

54. The method of claim 53, wherein said siRNA configured to inhibit expression of ROCK1/2 in said epithelial recipient cell comprises a siRNA according to SEQ ID NO. 1, and SEQ ID NO. 2.

55. The method of claim 51, wherein said ROCK1 inhibitor comprises a siRNA configured to inhibit expression of ROCK1 in said epithelial recipient cell.

56. The method of claim 55, wherein said siRNA configured to inhibit expression of ROCK1 in said epithelial recipient cell comprises a siRNA according to SEQ ID NO. 1.

57. The method of claim 46, wherein said target inhibitor comprises a target inhibitor selected from the group consisting of: a small-molecule, a small-inhibitory RNA (siRNA), a short hairpin RNA (shRNA), a bifunctional RNA, an antisense oligonucleotide, an antibody or functional fragment thereof, a ribozyme, a deoxyribozyme, an aptamer, a small molecule or gene therapy that knocks out a target gene.

58. The method of claim 46, wherein said target inhibitor is selected from Actinomycin D, HCl, Doxorubicin, or a combination of the same.
